(19) United States
(12) Patent Application Publication
(10) Pub. No.: US 2002/0053097 A1
(43) Pub. Date: May 2, 2002

(54) Transgenic plants containing heat shock protein

(76) Inventors: Susan Lindquist, Chicago, IL (US); Christine Queitsch, Chicago, IL (US); Elizabeth Vierling, Tuscon, AZ (US)

Correspondence Address: FULBRIGHT & JAWORSKI, LLP, 1301 MCKINNEY, SUITE 5100, HOUSTON, TX, 77010-3095, US

(21) Appl. No.: 09/812,350

(22) Filed: Mar. 20, 2001

Related U.S. Application Data
(60) Provisional application No. 60/190,769, filed on Mar. 20, 2000.
(60) Provisional application No. 60/198,116, filed on Apr. 18, 2000.

(51) Int. Cl. A01H 5/00; C12N 15/82

(52) U.S. Cl. 800/298; 800/278

(57) ABSTRACT

A transgenic plant having increased stress tolerance, such as thermotolerance, comprises a Hsp100 family nucleic acid sequence. The invention is also directed to methods of producing products from transgenic Hsp100 plants.

This nonprovisional application claims priority to U.S. Provisional Patent Application No. 60/190,769, filed Mar. 20, 2000, and to U.S. Provisional Patent Application No. 60/198,116, filed Apr. 18, 2000.

The work herein was supported by grants from the United States Government. The United States Government may have certain rights in the invention.

### FIELD OF THE INVENTION

This invention relates to transgenic plants which express a nucleic acid sequence of the Hsp101 family at increased levels, thereby allowing the plants to be tolerant to stresses such as heat.

### BACKGROUND OF THE INVENTION

Genetic systems which permit organisms to respond defensively to stress have been inferred from empiric observations. One of the major stresses which triggers a response from intact organisms, tissues or cultured cells, is temperature, both extreme heat and extreme cold. In this context “extreme” means temperature ranges that are undesirable for normal physiological functioning and/or survival of a particular genus and species. There appears to be an almost universal response of organisms to heat shock, that response being to produce a small number of proteins. When cells are exposed to mildly elevated temperatures, they respond by producing a small number of proteins called the heat-shock proteins, or hsps (See review by Lindquist, 1986, for a general treatment of the heat shock response and the review by Lindquist and Craig, 1988, for a detailed description of what is known about the functions of the heat shock proteins; Hemmingsen et al., 1988; Deshaies et al., 1988; Chirico et al., 1988; Kang et al., 1990; Cheng et al., 1989; Reading et al., 1989; Borkovich et al., 1989; Picard et al., 1990; Rothman et al., 1989.) This response is the most highly conserved genetic regulatory system known. In both eukaryotic and prokaryotic organisms, heat shock genes have been localized and found to be scattered among various chromosomal locations.

One of the most closely studied of these is the induction of HSPs (heat-shock proteins), which comprise several evolutionarily conserved protein families. All of the major HSPs (that is, those that are expressed at very high levels in response to heat and other stresses) have related functions: they ameliorate problems caused by protein misfolding and aggregation. However, each major HSP family has a unique mechanism of action. Some promote the degradation of misfolded proteins (Lon, ubiquitin, and various ubiquitin-conjugating enzymes); others bind to various types of folding intermediates and prevent them from aggregating (Hsp70 and Hsp60), and yet another promotes the reactivation of proteins that have already aggregated (Hsp100) (Parsell and Lindquist, 1993; Parsell and Lindquist, 1994b).

The heat-shock response was first discovered in the fruit fly, Drosophila melanogaster. Since then, it has been found in virtually all organisms, including bacteria, plants, warm and cold blooded vertebrates, protozoa, insects, sea urchins, slim molds, and fungi. (The single known exception is a few species of Hydra.) In multicellular organisms, the response is observed in virtually every tissue, and at every stage of development. The response can also be induced by a variety of other stress treatments, such as exposure to ethanol, anoxia, and heavy metal ions.

Exposing cells and organisms to mild stress, such as moderately warm temperatures and low concentrations of ethanol, also induces tolerance to more extreme stresses such as higher temperatures and higher concentrations of ethanol. Because of the general correlation between the induction of tolerance and the synthesis of heat shock proteins, for many years it has been postulated that heat shock proteins might play an important role in the acquisition of tolerance. However, it has been reported in several organisms that inhibiting the synthesis of heat shock proteins does not inhibit the induction of tolerance. (See review by Li, 1985 for more detailed discussion.) Genetic tests of the function of the heat shock proteins also suggested the proteins might not be involved in thermotolerance. In particular, several of the genes encoding heat shock proteins have recently been mutated. One of these mutations (in hsp26) has no affect on the ability of a cell to withstand high temperatures (Petko and Lindquist, 1986). Some of these mutations (e.g. mutations in hsp60, hsp70, and hsp82) affect the ability of cells to grow at normal temperatures and at moderately warm temperatures. For example, cells need to make hsp82 in order to live at any temperature, but they need even higher concentrations of the protein to live and grow at higher temperatures (Borkovich et. al., 1989). These mutations either do not affect the ability of an organism to tolerate extreme temperatures or actually increase its ability to survive at extreme temperatures. (For hsp70 mutation effects see Craig and Jacobsen, 1984.)

Mutations in another heat shock gene, ubiquitin, affect the ability of cells to survive chronic exposure to temperatures at the very upper end of their normal growth range, but again, these mutations produced cells which survived extreme temperatures as well as, or better than, the wild-type (Findlay and Varshevsky, 1987). Thus most of the heat shock proteins examined to date play vital roles in the cell at normal temperatures. Additionally, they help to extend the normal temperature growth range of a cell. In addition to being universal, these proteins appear to be highly conserved not only in their protein coding sequences, but also in their regulatory sequences. These findings suggest an evolutionary importance for the role of genes which encode these proteins. Ubiquitin and the hsp proteins may be complementary methods of dealing with a common stress problem, that is, the production of denatured protein aggregates in heat shocked cells.

The heat-shock response of Drosophila melanogaster, the organism in which the response was discovered, is the most well characterized among higher eukaryotes. The intensity of the Drosophila response is particularly striking and provides one of the best examples of a reversible, global redirection of macromolecular synthesis (Lewis et al., 1975; Chomyn et al., 1979; DiDomenico et al., 1982). Immediately after a shift from 25° C. (the normal growing temperature of Drosophila tissue culture cells) to 37° C. (a heat-shock inducing temperature) transcription is redirected from the synthesis of normal 25° C. mRNAs to the synthesis of heat-shock mRNAs, the most abundant of which is hsp70 mRNA (Ashburner, 1970; Tissieres et al., 1974; McKenzie et al., 1975; Spradling et al., 1975; McKenzie and Meselson, 1977). At the same time, pre-existing mRNAS are translationally repressed while newly transcribed heat-shock mRNAs are translated at very high rates (Mirault et al., 1978; Lindquist, 1980a; Scott and Purdue, 1981). This translational pattern persists as long as the temperature remains elevated. When the cells are returned to 25° C., heat-shock protein synthesis is repressed and normal protein synthesis is restored (DiDomenico et al., 1982a; Lewis, 1975; Chomyn et al., 1979).

In microorganisms such as the yeast Saccharomyces cerevisiae and the bacterium Escherichia coli, heat shock proteins are also induced very rapidly after a shift to high temperatures. However, the synthesis of normal cellular proteins is not as severely impaired and during continued exposure to moderately high temperatures (i.e., 37°−40° C.) growth may resume after heat shock proteins have accumulated. At yet higher temperatures, heat-shock protein synthesis continues until, eventually, cells begin to die.

From these results and subsequent studies on a number of other organisms, it has been suggested that the heat response is transient in most organisms. When organisms are returned to normal temperatures after brief exposure to high temperatures, normal patterns of protein synthesis are restored and growth resumes. When maintained at moderately warm temperatures, growth resumes after a temporary pause. When maintained at higher temperatures, heat shock protein synthesis continues until the cells slowly begin to die. The metabolic state or developmental stage of the cell may affect the response.

Examples of Heat Induced Proteins

Previously reported heat shock proteins and other similar proteins appear to play essential roles in growth and metabolism at normal temperature (e.g., hsp70, hsp60, hsp62). Heat shock proteins have been assigned names corresponding to their approximate apparent molecular weight. Hsp70 is the most highly conserved of the hsp proteins. The complete amino acid sequence of hsp70 proteins from various organisms is presented in a review by Lindquist (1986).

Many differences among species are due to homologous substitutions in hsp70. Other differences may represent responses during evolution to the necessity to survive in ecological niches having different temperatures. In addition, in Drosophila, Saccharomyces, and all eukaryotes analyzed, hsp70 genes appear to belong to a multi-gene family whose members respond to temperature in different ways; some members are synthesized at low temperatures and some are targeted to different cellular compartments.

In another size range, all eukaryotic cells studied to date appear to produce a prominent heat-shock protein in the range of 82-90 kd e.g., hsp 82 and 90. Hsp90 has been found to be a major component of several steroid receptor complexes. It also complexes with various oncogenic protein kinases.

Most eukaryotic cells produce proteins in the range of 100 to 110 kD after exposure to high temperatures (Lindquist and Craig, 1988). To date, these proteins have been studied only in mammalian and yeast cells. In mammalian cells, investigations focus on the mammalian 110 kD protein concentrates in the nucleoli of these cells. (Subjeck et al, 1983). Interestingly, the precise staining pattern obtained with anti-hsp 110 antibody is dynamic, changing with growth state, nutritional conditions and heat shock (Subjeck et al, 1983; Shyy, et al, 1986; Welch and Suhan, 1985). Prior to the present invention, the relationships between the mammalian 110 kD protein, the yeast hsp 104 protein, and the high molecular weight heat shock proteins of other organisms were unknown.

There is also a large category of smaller molecular weight hsp proteins which, although varying in size and number in different species, are said to be homologous, i.e. to show identity for certain percentages of their amino acid sequences. For example, in Drosophila, designations for such proteins are hsp22, 23, 27, and 28.

Although all organisms synthesize HSPs in response to heat, the balance of proteins synthesized and the relative importance of individual HSP families in tolerance vary greatly among organisms. For example, in the yeast Saccharomyces cerevisiae, a member of the Hsp100 (ClpB/C) family, Hsp104, is strongly expressed in the nuclear/cytoplasmic compartment in response to stress and plays a particularly pivotal role in tolerance to extreme conditions (Sanchez et al., 1992; Parsell et al., 1994). Yeast cells expressing Hsp104 survive exposure to high temperatures or high concentrations of ethanol a thousand- to ten thousand-fold better than cells not expressing Hsp104. Members of the Hsp100 family also play critical roles in the stress tolerance of bacterial cells (Schirmer et al., 1996), including photosynthetic organisms like cyanobacteria (Eriksson and Clarke, 1996). In contrast, the fruit fly Drosophila melanogaster does not even make a protein of this type in response to stress. Instead, the induction of Hsp70 plays the central role in stress tolerance in this organism (Solomon et al., 1991; Welte et al., 1993). Determining which proteins play the most crucial roles in stress tolerance in different types of organisms requires genetic analysis. Among organisms amenable to such analysis, higher plants present a particularly interesting subject. First, their immobility limits the range of their behavioral responses to stress and places a particularly strong emphasis on cellular and physiological mechanisms of protection. Second, their natural environments subject them to wide variations in temperature, seasonally and diurnally. Third, they are developmentally complex and the nature of the stresses to which they are exposed, as well as their responses to stress, are likely to vary in different tissues of the same organism at the same time. Even for a particular organ, for example among leaves, temperatures can vary dramatically with position on the plant (sun exposure) and can change abruptly with a shift in shading. Finally, the ability to withstand heat stress, especially in combination with water stress, may be of great importance in agricultural productivity (Levitt, 1980; Frova, 1997). Surprisingly, the critical factors conferring temperature tolerance in higher plants are still poorly understood. There is evidence which suggests that HSPs, as a general class, are likely to play some role. Several studies have correlated the induction of HSPs by mild heat stress with the induction of tolerance to much more severe stress (Ougham and Howarth, 1988; Vierling, 1991; Howarth and Skot, 1994). In addition, overexpression of certain transcriptional regulators of HSP expression, HSF1 and HSF3, causes plants to constitutively express at least some HSPs and produces somewhat higher levels of basal thermotolerance (Lee et al., 1995; Prändl et al., 1998).

Several members of the Hsp100 family have been identified in higher plants, and their genes have been cloned (Lee et al., 1994; Schirmer et al., 1996; Boston et al., 1996; Wells et al., 1998). Like many other HSP families, the Hsp100 protein family comprises both heat inducible and constitutive members. Between plants, bacteria and yeast, these heat inducible members are more closely related to each other than they are to their own constitutively expressed relatives (Schirmer et al., 1996). Their sequence homology and similar patterns of induction suggest a related function in stress tolerance. Moreover, the Arabidopsis, soybean, wheat, and tobacco Hsp100 homologs can at least partially restore thermotolerance to yeast cells carrying an hsp104 deletion (Lee et al., 1994; Schirmer et al., 1994; Wells et al., 1998).

Tolerance

Mild heat pretreatments are able to effect thermotolerance. If cells are shifted directly to an extreme temperature, lethality is likely. However, at a more moderate elevated temperature, hsp synthesis is induced. If the cells are later exposed to extreme temperatures, there is a dramatic increase in survival compared to the initial lethality response. At stages in development in which hsps cannot be induced, organisms are extremely sensitive to heat and thermotolerance cannot be induced. This phenomenon has been observed in a wide variety of plants, animals, fungi, and bacteria.

These observations suggest that hsps may play a role in thermotolerance. This proposed function, however, has been controversial (Riabawal et al., 1988; McAlister et al., 1980; Li and Laszlo, 1982; Hall, 1983; Widelity et al., 1986; Carper et al., 1987). Mutations in most heat shock protein genes do not compromise thermotolerance. Also, certain inhibitors which block the synthesis of hsps have been reported not to interfere with thermotolerance.

Stresses Other than Heat

Interestingly, in many organisms such as Drosophila, chirlurus, and yeast, proteins found initially by their induction due to heat shock are similar in structure to those found to be induced by a wide variety of other stresses, for example, alcohol, anoxia, and metals such as cadmium and sodium arsenate. A compilation of the various inducers is provided by Nover (1984), and a summary is presented in Lindquist (1986).

Some of these inductions have only been tested in a small number of organisms and may be unique to particular biological circumstances, but it is clear that others are universal, or nearly universal. Among the most common inducers are ethanol and heavy metal ions. The assumption that these inductions have biological significance rests upon the observation that they are generally associated with increased tolerance, both to the inducing agent itself and to other types of stress. For example, pre-treatments with moderate concentrations of ethanol induce tolerance to yet higher concentrations of ethanol and, at the same time, tolerance to high temperatures. In a complementary fashion, mild heat treatments induce tolerance to both higher temperatures and to high concentrations of ethanol.

This phenomenon is called cross tolerance, and it has been postulated that the heat-shock proteins are responsible for it. Whether individual hsps are of the same relative importance in tolerance to different types of stress was unclear prior to the work described in this invention. Indeed, some investigators have questioned whether hsps play any role at all in tolerance to certain types of stress.

The nucleotide sequences responsible for induction of one of the hsp70 genes of human cells that is induced by heat, cadmium, the adenoma virus Ela protein, and the addition of serum to serum starved cells has been mapped. In this system there appear to be different sequences responsible for the cadmium and heat shock induction, than for the serum stimulation and possibly the viral induction.

Regulation of Stress Response

A striking feature of heat-shock gene expression is that the responses of different organisms, and, indeed, of different cell types within an organism are regulated in different ways. In E. coli and in yeast the response is controlled primarily at the level of transcription. In Drosophila regulation is exerted on transcription, translation, and message turnover. 5′ upstream sequences are postulated to serve in the transcriptional activation; the activation site maps to a consensus element shared by all eukaryotes. A synthetic promoter sequence derived from this consensus region seems to be sufficient for at least partly heat-inducible transcription in heterologous systems. The consensus element sequence (HSE) and a heat-shock transcription factor (HSTS) that binds to the HSE, have been isolated and characterized. Most heat shock genes appear to contain multiple consensus elements.

Heat shock proteins are also regulated at the translational level in most organisms. In Drosophila the translation of heat shock proteins depends upon sequences in the 5′ region. Few inducers are able to effect this translational response. When hsps have accumulated in sufficient quantities, the translation of pre-existing non-heat shock messages is restored and the translation of heat-shock messages is repressed.

Despite many intriguing empiric observations of stress response, major unanswered questions remain about stress-response systems, including how they function to exert their protective and tolerance-inducing effects, and what is the extent of inducible stress and specific stress ranges. The specific protective mechanisms of stress response have proven elusive quarry. Components of the systems have generally not been identified, isolated and purified.

Understanding these mechanisms and identifying, isolating and purifying their components, would provide methods for controlling the responses of an organism to its environment. For example, teratogens that cause developmental malformations also induce hsps. This may be the cause of the malformations; alternatively, the well-known variation in individual responses to teratogens may reflect differences in their genetic stress response. If the latter is the case, malformation risk may be reduced by enhancing the stress response systems. The present invention elucidates some of these genetic stress-response mysteries and discloses the isolation, purification and manipulation of stress response system components. Clinical and commercial uses of stress response systems are described which have not been previously developed.

### SUMMARY OF THE INVENTION

In an embodiment of the present invention there is a transgenic plant comprising a genetic construct wherein the construct comprises a promoter, wherein the promoter is operatively linked to a nucleic acid sequence encoding a plant Hsp100 family amino acid sequence.

In a specific embodiment of the present invention the plant Hsp100 family amino acid sequence is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29. In another specific embodiment the nucleic acid sequence encoding the plant Hsp100 family amino acid sequence is endogenous to the transgenic plant.

In an additional specific embodiment the nucleic acid sequence has sequence similarity with a sequence selected from the group consisting of the GenBank accession numbers SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, and SEQ ID NO:49. In another specific embodiment of the present invention the transgenic plant is selected from the group consisting of a cereal, a grass, an ornamental plant, a crop plant, a food plant, an oil-producing plant, a synthetic product-producing plant, an environmental waste absorbing plant, an alcohol plant, a medicinal plant, a recreational plant and an animal feed plant. In an additional embodiment of the present invention the transgenic plant is selected from the group consisting of cotton, canola, soybean, corn, wheat, tobacco, sorghum and Arabidopsis thaliana.

In an additional specific embodiment the promoter is selected from the group consisting of a constitutive promoter and an inducible promoter. In a further specific embodiment the constitutive promoter is selected from the group consisting of a 35S cauliflower mosaic virus promoter, a CaMV-35Somega promoter, an Arabidopsis ubiquitin UBQ1 promoter, and a barley leaf thionin BTH6 promoter. In an additional specific embodiment the consitutive promoter is a 35S cauliflower mosaic virus promoter.

The inducible promoter in another specific embodiment is heat inducible. In a specific embodiment the inducible promoter is selected from the group consisting of a heat shock protein promoter, a heat shock transcription factor promoter, a chaperonin promoter, an A1494 promoter, a rice genomic metallothionein-like gene (rgMT) promoter a ubiquitin promoter, an FLP promoter, an Oryza sativa metallothionein like gene-2 (OsMT-2) promoter, a Glycine max STI1 (gmsti) promoter, a synthetic heat shock promoter and a TCH gene promoter.

In an embodiment of the present invention there is a method of increasing thermotolerance of a plant comprising the steps of preparing a transgenic plant comprising a genetic construct wherein the construct comprises a promoter, wherein the promoter is operatively linked to a nucleic acid sequence encoding a plant Hsp100 family amino acid sequence; and exposing the transgenic plant to a heat pretreatment. In a specific embodiment the plant is a seedling.

In another embodiment of the present invention there is a method of producing a crop comprising the steps of preparing a crop plant wherein the plant comprises a genetic construct which comprises a promoter operatively linked to a nucleic acid sequence encoding a plant Hsp100 family amino acid sequence, growing the crop plant in an environment which produces heat stress; and extracting the crop from the transgenic cotton plant. In a specific embodiment the crop plant is selected from the group consisting of cotton, tobacco, corn, sorghum, rice, wheat, peanut, soybean and canola.

It is an object of the present invention to provide a method of producing oil from a plant comprising the steps of preparing a transgenic oil-producing plant wherein the plant comprises a genetic construct comprising a promoter operatively linked to a nucleic acid sequence encoding a plant Hsp100 family amino acid sequence; growing the transgenic oil-producing plant in an environment which produces heat stress; and extracting the oil from the transgenic oil-producing plant. In a specific embodiment the oil-producing plant is selected from the group consisting of canola, corn, peanut, olive and soybean.

It is an object of the present invention to provide a method of producing a synthetic product from a plant comprising the steps of preparing a synthetic product-producing plant wherein the plant comprises a genetic construct comprising a promoter operatively linked to a nucleic acid sequence encoding a plant Hsp100 family amino acid sequence; growing the synthetic product-producing plant in an environment which produces heat stress; and preparing the synthetic product from the synthetic product-producing plant.

It is an object of the present invention to provide a method of making an environmental waste absorbing plant comprising the steps of preparing a transgenic environmental waste absorbing plant wherein the plant comprises a genetic construct comprising a promoter operatively linked to a nucleic acid sequence encoding a plant Hsp100 family amino acid sequence; growing the transgenic oil-producing plant in an environment which produces heat stress; and removing the environmental waste from said environment.

It is an object of the present invention to provide a method of making a medicinal plant comprising the steps of preparing a transgenic medicinal plant wherein the plant comprises a genetic construct comprising a promoter operatively linked to a nucleic acid sequence encoding a plant Hsp100 family amino acid sequence; growing the medicinal plant in an environment which produces heat stress; and preparing a medicament from the medicinal plant.

In another embodiment of the present invention there is a method of making animal feed from a plant comprising the steps of preparing a transgenic animal feed-producing plant wherein the plant comprises a genetic construct comprising a promoter operatively linked to a nucleic acid sequence encoding a plant Hsp100 family amino acid sequence; growing the animal feed-producing plant in an environment which produces heat stress; and preparing the animal feed from the animal feed-producing plant. In a specific embodiment the plant is selected from the group consisting of sorghum, soybean, wheat and corn.

In another embodiment of the present invention there is a method of making alcohol from a plant comprising the steps of preparing an ethanol-producing plant wherein the plant comprises a genetic construct comprising a promoter operatively linked to a nucleic acid sequence encoding a plant Hsp100 family amino acid sequence; growing the alcohol plant in an environment which produces heat stress; and preparing the alcohol from the alcohol plant.

In another embodiment of the present invention there is a method of utilizing a recreational plant comprising the steps of preparing a recreational plant wherein the plant comprises a genetic construct comprising a promoter operatively linked to a nucleic acid sequence encoding a plant Hsp100 family amino acid sequence; growing the recreational plant in an environment which produces heat stress; and utilizing the plant for recreational purposes. In a specific embodiment the recreational plant is a grass.

In another embodiment of the present invention there is a seed from a transgenic plant comprising a genetic construct wherein the construct comprises a promoter, wherein the promoter is operatively linked to a nucleic acid sequence encoding a plant Hsp100 family amino acid sequence.

Other and further objects, features and advantages would be apparent and eventually more readily understood by reading the following specification and by reference to the company drawing forming a part thereof, or any examples of the presently preferred embodiments of the invention are given for the purpose of the disclosure.

### BRIEF DESCRIPTION OF THE DRAWINGS

FIG. 1 shows western analysis of representative samples from vector control lines (No-V1 and Col-V1), antisense lines (No-AS1 and No-As2), co-suppression lines (Col-SUP1 and Col-SUP2), and constitutive expression lines (No-C1, Col-C1 and Col-C2).

FIG. 2 illustrates representative examples of plants from two vector control lines (No-V1, Col-V1), an antisense line (No-AS1), a co-suppression line (Co-SUP1), and a constitutive expression line (Col-C1) at fourteen days (top), three weeks (middle), and five weeks (bottom) of development after growth in continuous light.

FIG. 3 shows fourteen-day-old seedlings grown at 22° C. which were given a conditioning pretreatment at 38° C. for 90 min, immediately subjected to a severe heat shock at 45° C. for 2 hr, and then returned to 22° C. for recovery.

FIG. 4 demonstrates seeds germinated on plates at 22° C. for 30 min (after seed plating), 30 hr, 36 hr, 48 hr, or 72 hr which were exposed to 47° C. for 2 hr (HS).

FIGS. 5A through 5B demonstrate expression levels of Hsp101 in seeds of antisense plants. FIG. 5A shows Hsp101 and Hsp17.6 expression in vector control versus antisense seeds. FIG. 5B shows seedlings of all five antisense lines and two control lines which were germinated for 30 hr and then exposed to 47° C. for 2 hr.

FIG. 6 illustrates fourteen-day-old plants which were grown at 22° C., shifted directly to 45° C. for 30, 45, or 60 min and returned to 22° C.

FIGS. 7A through 7C show constitutive expression of Hsp101 in three-day-old seedlings. FIG. 7A illustrates analysis of Hsp101 expression in three-day-old seedlings. FIG. 7B shows seeds of vector controls and constitutive lines which were germinated for three days, heat-shocked at 47° C. for 30 min and then returned to 22° C. FIG. 7C demonstrates a representative plate (detail) with the same transgenic lines from the same experiment as in FIG. 7B which was photographed ten days after heat shock.

FIG. 8 illustrates the percentage of Arabidopsis seed germination after heat treatment of wild type (Col) vs. an insertional Hsp101 mutant.

### DESCRIPTION OF THE INVENTION

It will be readily apparent to one skilled in the art that various embodiments and modifications may be made in the invention disclosed herein without departing from the scope and spirit of the invention.

I. Definitions

As used in the specification, “a” or “an” may mean one or more. As used in the claim(s), when used in conjunction with the word “comprising”, the words “a” or “an” may mean one or more than one. As used herein “another” may mean at least a second or more.

The term “constitutive promoter” as used herein is defined as a nucleic acid sequence which regulates transcription of an associated nucleic acid sequence and which promotes transcription in the absence of an inducing stimulus. Examples of constitutive promoters are the 35S Cauliflower Mosaic Virus promoter, the CaMV-35Somega promoter, the Arabidopsis ubiquitin UBQ1 promoter, and the barley leaf thionin BTH6 promoter.

The term “genetic construct” as used herein is defined as a nucleic acid sequence comprising a synthetic arrangement of at least two nucleic acid segments for the purpose of creating a transgenic plant. In a specific embodiment, one nucleic acid segment is a regulatory sequence and another nucleic acid segment encodes a gene product. In a further specific embodiment, the gene product is a Hsp100 amino acid sequence.

The term “heat stress” as used herein is defined as the exposure to temperatures at least in the upper range of natural growth temperatures for the specific plant. In a specific embodiment, the heat stress is applied as a heat pretreatment. In a specific embodiment, the heat pretreatment or the environment which produces heat stress of the present invention may be generated by natural means, such as by sunlight, or by artificial means, such as by an electronically-generated or fuel-generated heating source.

The term “Hsp100 family” as used herein is defined as an amino acid sequence which has an overall amino acid homology at the protein level of about at least 40% to Arabidopsis thaliana Hsp100, and this includes the nucleic acid sequences which encode those proteins. In an alternative embodiment, the family is directed to having sequence similarity to the yeast Hsp104 sequence. In a specific embodiment, the sequence is a plant sequence.

In preferred embodiments, the proteins which are functionally and structurally related to Arabidopsis Hsp101. A skilled artisan recognizes that Arabidopsis Hsp101 belongs to the class 1 Hsp100 s/Clb protein family. Members of this class contain two nucleotide binding domains, flanked by amino-terminal, middle (or spacer) and carboxy-terminal regions. The two nucleotide binding domains are highly conserved in all members identified to date, but have very different amino acid sequences (except both contain Walker A and B nucleotide binding elements.) Subfamilies of class 1 Hsp100 's are distinguished mainly by the size of the spacer region (shortest class: A; intermediate: D, C; longest: B). Based on this structural feature and sequence homology at the amino acid level, it is preferred to contain within the scope of the present invention proteins which would be classified into classes B (spacer region being about 170 to about 200 residues), C and D (spacer about 100 to about 120 residues), or proteins which have a spacer region anywhere from 100 to over 200 residues, thereby excluding the classl A subfamily. The overall amino acid homology of proteins to Arabidopsis Hsp101 is preferably about 40% and higher. Examples of specific amino acid sequences which meet this criteria include (as represented by their GenBank Accession numbers): P53533 (SEQ ID NO:1); CAA69406 (SEQ ID NO:2); BAA04506 (SEQ ID NO:3); CAA40846 (SEQ ID NO:4); CAA53534 (SEQ ID NO:5); AAB49540 (SEQ ID NO:6); AAA50477 (SEQ ID NO:7); and P31543 (SEQ ID NO:8). The corresponding nucleic acid sequences for these amino acid sequences include: U20646 (SEQ ID NO:9); Y08238 (SEQ ID NO:10); D17582 (SEQ ID NO:11); X57620 (SEQ ID NO:12); X75930 (SEQ ID NO:13); U43536 (SEQ ID NO:14); M67479 (SEQ ID NO:15); M92325 (SEQ ID NO:16). In specific embodiments, the overall amino acid homology of proteins to Arabidopsis Hsp101 is about 41%, about 42%, about 43%, about 44%, about 45%, about 47%, about 50%, about 55%, about 60%, about 65%, about 70%, about 75%, about 80% and higher.

Examples of other amino acid sequences useful in the present invention include: P42730 (SEQ ID NO:17); AAD22629 (SEQ ID NO:18); AAC83689 (SEQ ID NO:19); AAA66338 (SEQ ID NO:20); AAD25223 (SEQ ID NO:21); AAF01280 (SEQ ID NO:22); AAC83688 (SEQ ID NO:23); CAB46061 (SEQ ID NO:24); CAB08073 (SEQ ID NO:25); CAA86116 (SEQ ID NO:26); AAD33606 (SEQ ID NO:27); AAD26530 (SEQ ID NO:28); AAF91178 (SEQ ID NO:29)

Examples of other nucleic acid sequences useful in the present invention include: U13949 (SEQ ID NO:30); AF218796 (SEQ ID NO:31); L35272 (SEQ ID NO:32); AF083343 (SEQ ID NO:33); AF174433 (SEQ ID NO:34); AF133840 (SEQ ID NO:35); AF083327 (SEQ ID NO:36); AF077337 (SEQ ID NO:37); AF203700 (SEQ ID NO:38); AF097363 (SEQ ID NO:39); AF083344 (SEQ ID NO:40); U20646 (SEQ ID NO:41); AF016634 (SEQ ID NO:42); AF043539 (SEQ ID NO:43); AF023422 (SEQ ID NO:44); U40604 (SEQ ID NO:45); AJ224159 (SEQ ID NO:46); AF022909 (SEQ ID NO:47); Z94053 (SEQ ID NO:48); and Z38058 (SEQ ID NO:49). In specific embodiments, sequences utilized in generating the transgenic plants of the present invention have an overall identity of about 25%, about 26%, about 27%, about 28%, about 29%, about 30%, about 35%, about 40%, about 45%, about 50%, about 55%, about 60%, about 65%, about 70%, about 75%, about 80%, about 85%, about 90%, about 95%, and about 100%.

Examples of plants which have sequence similarity to these sequences are higher plants including cotton, canola, soybean, corn, wheat, tobacco, Arabidopsis thaliana, peanut and sorghum.

The term “induced thermotolerance” as used herein is defined as the ability of an organism to survive a normally lethal temperature if it is first conditioned by pretreatment at a milder temperature.

The term “inducible promoter” as used herein is defined as a nucleic acid sequence which regulates transcription of an associated nucleic acid sequence and which is subject to control by an external stimulus. Examples of such stimuli are heat, cold, touch, wind, hormones, growth factors, steroids, light, vibration and sound. In a preferred embodiment, the inducible promoter is heat inducible. Examples of heat inducible promoters include a heat shock protein promoter, a heat shock transcription factor promoter, a chaperonin promoter, a ubiquitin promoter, an A1494 promoter, a rice genomic metallothionein-like gene (rgMT) promoter, an FLP recombinase promoter, an Oryza sativa metallothionein like gene-2 (OsMT-2) promoter, a Glycine max STI1 (gmsti) promoter, a synthetic heat shock promoter and a TCH gene promoter. A synthetic heat shock promoter as herein defined is a non-naturally occurring promoter such as described in Strittmatter and Chua, (1987), wherein more than one heat shock-like element or heat shock element are used to provide regulation of a nucleic acid sequence upon exposure to heat.

The term “medicament” as used herein is defined as a medicine, vitamin or health-improving chemical or composition.

The term “promoter” as used herein is defined as a nucleic acid sequence which regulates expression of another nucleic acid sequence. The promoter may include enhancers or other elements which affect the initiation of transcription, the beginning site of transcription, levels of transcription, the ending site of transcription, or any postprocessing of the resulting ribonucleic acid. The promoter may be inducible or constitutive.

The term “stress” as used herein is defined as any factor or agent which is potentially deleterious to the cell, generally being a value outside of the physiological range at which the organism is able to function. Heat stress, for example, is defined as those temperatures that are at least at the upper end of the organism's natural growth range. Examples of stresses to a plant include heat, drought, chilling, freezing, exposure to pathogen, pH, exposure to alcohols, exposure to metals, disease, excess moisture, salt, and oxidative stress.

The term “transcription” as used herein is defined as the generation of an RNA molecule from a DNA template.

II. The Present Invention

As disclosed herein, the present invention is directed to methods and compositions regarding stress tolerance in a plant utilizing a Hsp100 family sequence. A skilled artisan recognizes that a multitude of plants would benefit from containing a sequence which imparts resistance to stresses, such as heat. A skilled artisan recognizes that the sequence to be introduced into the plant may be endogenous to the plant or may be from another plant. Although one skilled in the art could apply the methods and compositions as described herein to any species, specific examples include the following: cotton; rice; barley; oats; canola; soybean; corn; wheat; rye; tobacco; sorghum; Arabidopsis thaliana; sunflower; alfalfa; tomato; potato; sugar beet; cassava; broccoli; cauliflower; peanut; olive tree; grass, such as St. Augustine, hybrid Bermuda grass, rye grass, Zoysiagrass, turfgrass, and coastal Bermuda grass; flowering plants, such as roses carnations, daisies, orchids, tulips, and irises; palms; ferns; woody plants, shrubs, ficus, evergreen, ivy; grapes; hops; aloe vera; opium poppy; sweet potatoes; yams; Echinacea; witch hazel; and Gingko biloba; trees; ornamentals; vegetable-bearing plants; and fruit-bearing plants.

The stress response system of the present invention comprises a regulatory system which is capable of enabling structural genes to be expressed. At least one promoter or regulatory area is necessary for operation of the genetic stress response system. In an illustrative embodiment, this promoter is inducible by at least one environmental stress factor. Factors found to be inducers include heat, ethanol, cadmium, sodium arsenite, and nitrogen starvation. Alternatively, heterologous promoters, for example promoters induced by hormones or sugars, are suitable promoters. In an illustrative embodiment, a heterologous promoter is a hormone, such as deoxycorticosterone or deacylcortivazol. Sugars such as galactose or glucose may also act as inducers. The promoter may also be constitutive. Of great use to commercial and clinical applications of stress protector protein encoding genes is that by placing the coding sequences under the control of various promoters, e.g., the galactose regulated promoter gal 1, the thennotolerance of cells varies with the presence or the absence of the sugar. That is, it is easily subject to heterologous control with exposure to stress.

In an illustrative embodiment, these proteins comprise the family designated the hsp100 proteins. This stress-response system has been identified, isolated, purified, manipulated and applied in the present invention. It has some similarities to other stress response systems, but it differs from all others in two respects. First, the hsp100 proteins are the only proteins to date with a demonstrated function in protecting organisms from several different types of extreme stress. Second, they are apparently not necessary for cellular functioning except when stress is present, that is, the are not a vital component of normal physiological functioning. An advantage of this property is that components of this system can be altered without disturbing other cellular functions which must remain intact for normal life.

Proteins in the hsp100 family have apparent molecular weights in approximately the range 80-120 kd as determined by SDS polyacrylamide gel electrophoresis. The hsp100 proteins generally have an amino acid sequence of about 900 residues and show particularly high levels of homology in regions surrounding the two nucleotide binding sites.

Proteins that are expressed by the hsp100 structural genes are members of a family of proteins designated here as hsp100 because the apparent molecular weights of the most prominent heat-inducible members are in the 80-120 kd range. They also share other properties and sequence homologies with the Clp family.

The hsp100 family of proteins is very highly conserved, comparable to other hsps families. The family most likely plays a major role in thermotolerance in all organisms. Proteins in this family exhibit similarities to the ClpA protein but are even more highly homologous to ClpB protein of E. coli. Clp family members identified by sequence homology appear to be mitochrondrial. This may be a characteristic of the family. They are likely to be chaperone proteins that facilitate the export of proteins needed for stress-response, directly or indirectly establishing the correct protein assembly. A function of ClpB type proteins may be to protect protein from denaturation when stressed.

That a single family of proteins plays such a pivotal role in protecting organisms against the toxic effects of disparate stresses such as heat, ethanol, and arsenite, as well as against the damage that accumulates during long term storage at low temperatures, suggests that tolerance is mediated under these different conditions through a common biochemical pathway. By inference, it also suggests that the lethal lesions induced by such exposures are similar.

This invention does not relate only to a transgenic plant comprising a specific nucleic acid sequence, but rather to a transgenic plant comprising a nucleotide sequence homologous and functionally equivalent to an Hsp100 sequence or the biologically functional equivalent thereof. In an illustrative embodiment, the nucleotide sequence segment contains bases capable of encoding for an amino acid sequence sufficient to protect an organism or a cell against heat. This would include at least one nucleotide binding site. In an illustrative embodiment, the nucleic acid segment may be composed of DNA, for example, that encodes hsp100, a sequence of about 3.6 kb.

A recombinant vector for the generation of the genetic construct of the present invention may be produced by standard methods well known to those skilled in the art. The vector generally includes a nucleic acid segment, the segment capable of encoding at least one stress response protein. In an illustrative embodiment, the segment corresponds to the bases encoding amino acids 150 to 400 or amino acids 550 to 750, which are highly conserved regions including the nucleotide binding domains. The recombinant segment will generally be under the control of an effective promoter, as disclosed herein. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species pBR 322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own proteins.

A promoter is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The promoters most commonly used in recombinant DNA construction include the B-lactamase (penicillinase) and lactose promoter systems and a tryptophan (trp) promoter system. While these are the most commonly used, other microbial promoters have been discovered and utilized, and details concerning their nucleotide sequences have been published, enabling a skilled worker to ligate them functionally with plasmid vectors. However, it is an object of the present invention that the genetic construct contains a promoter which is consitutive or inducible, as discussed herein.

Methods of preparing nucleic acid segments which comprise at least the functional stress response portion of the coding sequence include obtaining genomic nucleic acids from eukaryotic or prokaryotic cells which comprise at least one coding region capable of expressing an active stress response protein, preparing recombinant clones which include at least one of the coding regions for the stress response protein, and selecting clones which comprise the desired amplified nucleic acid segment. Amplification may be accomplished by the polymerase chain reaction. Host cells will generally comprise in addition to the genetic construct for stress response, a promoter which provides for transcription of the gene and a translation initiative site which provides for expression of the protein from the gene transcript. Host cells may be eukaryotic cells, for example, yeast or human cells, or bacterial cells, for example, a lactobacillus or E. coli.

One of the important properties of the stress response system disclosed herein is to be able to induce tolerance to stress factors. This phenomenon refers to increasing the cell's or organism's ability to survive severe stress treatments which would otherwise be injurious causing the organism to produce the stress-protective protein.

The expression of a protein from the family hsp100 induced by stress, need not be induced by the same stress for which protection is being sought. Alternatively, the protein could be induced by other specific inducers that the recombinant gene is engineered to respond to, such as a sugar or a hormone. In another exemplary embodiment, the organism is engineered to produce the protein in the absence of specific inducers. This should result in higher constitutive or basal thermotolerance.

One of the most exciting and applicable uses for the stress response system is to control the response of an organism, such as a plant, to heat. In this application, a genetic construct is prepared comprising a heat or stress response gene capable of being expressed, operatively linked to a genetic promoter which is inducible by the heat. The genetic construct is introduced into the organism. The organism is then exposed to at least the level of heat capable of inducing expression of the heat or stress response gene.

III. Site-Specific Integration

It is specifically contemplated by the inventors that one could employ techniques for the site-specific integration or excision of transformation constructs prepared in accordance with the instant invention. An advantage of site-specific integration or excision is that it can be used to overcome problems associated with conventional transformation techniques, in which transformation constructs typically randomly integrate into a host genome in multiple copies. This random insertion of introduced DNA into the genome of host cells can be lethal if the foreign DNA inserts into an essential gene. In addition, the expression of a transgene may be influenced by “position effects” caused by the surrounding genomic DNA. Further, because of difficulties associated with plants possessing multiple transgene copies, including gene silencing, recombination and unpredictable inheritance, it is typically desirable to control the copy number of the inserted DNA, often only desiring the insertion of a single copy of the DNA sequence.

Site-specific integration or excision of transgenes or parts of transgenes can be achieved in plants by means of homologous recombination (see, for example, U.S. Pat. No. 5,527,695, specifically incorporated herein by reference in its entirety). Homologous recombination is a reaction between any pair of DNA sequences having a similar sequence of nucleotides, where the two sequences interact (recombine) to form a new recombinant DNA species. The frequency of homologous recombination increases as the length of the shared nucleotide DNA sequences increases, and is higher with linearized plasmid molecules than with circularized plasmid molecules. Homologous recombination can occur between two DNA sequences that are less than identical, but the recombination frequency declines as the divergence between the two sequences increases.

Introduced DNA sequences can be targeted via homologous recombination by linking a DNA molecule of interest to sequences sharing homology with endogenous sequences of the host cell. Once the DNA enters the cell, the two homologous sequences can interact to insert the introduced DNA at the site where the homologous genomic DNA sequences were located. Therefore, the choice of homologous sequences contained on the introduced DNA will determine the site where the introduced DNA is integrated via homologous recombination. For example, if the DNA sequence of interest is linked to DNA sequences sharing homology to a single copy gene of a host plant cell, the DNA sequence of interest will be inserted via homologous recombination at only that single specific site. However, if the DNA sequence of interest is linked to DNA sequences sharing homology to a multicopy gene of the host eukaryotic cell, then the DNA sequence of interest can be inserted via homologous recombination at each of the specific sites where a copy of the gene is located.

DNA can be inserted into the host genome by a homologous recombination reaction involving either a single reciprocal recombination (resulting in the insertion of the entire length of the introduced DNA) or through a double reciprocal recombination (resulting in the insertion of only the DNA located between the two recombination events). For example, if one wishes to insert a foreign gene into the genomic site where a selected gene is located, the introduced DNA should contain sequences homologous to the selected gene. A single homologous recombination event would then result in the entire introduced DNA sequence being inserted into the selected gene. Alternatively, a double recombination event can be achieved by flanking each end of the DNA sequence of interest (the sequence intended to be inserted into the genome) with DNA sequences homologous to the selected gene. A homologous recombination event involving each of the homologous flanking regions will result in the insertion of the foreign DNA. Thus, only those DNA sequences located between the two regions sharing genomic homology become integrated into the genome.

Although introduced sequences can be targeted for insertion into a specific genomic site via homologous recombination, in higher eukaryotes homologous recombination is a relatively rare event compared to random insertion events. In plant cells, foreign DNA molecules find homologous sequences in the cell's genome and recombine at a frequency of approximately 0.5-4.2×10⁻⁴. Thus any transformed cell that contains an introduced DNA sequence integrated via homologous recombination will also likely contain numerous copies of randomly integrated introduced DNA sequences. Therefore, to maintain control over the copy number and the location of the inserted DNA, these randomly inserted DNA sequences can be removed. One manner of removing these random insertions is to utilize a site-specific recombinase system. In general, a site specific recombinase system consists of three elements: two pairs of DNA sequence (the site-specific recombination sequences) and a specific enzyme (the site-specific recombinase). The site-specific recombinase will catalyze a recombination reaction only between two site-specific recombination sequences.

A number of different site specific recombinase systems could be employed in accordance with the instant invention, including, but not limited to, the Cre/lox system of bacteriophage P1 (U.S. Pat. No. 5,658,772, specifically incorporated herein by reference in its entirety), the FLP/FRT system of yeast (Golic and Lindquist, 1989), the Gin recombinase of phage Mu (Maeser and Kahmann, 1991), the Pin recombinase of E. coli (Enomoto et al., 1983), and the R/RS system of the pSR1 plasmid (Araki et al., 1992). The bacteriophage P1 Cre/lox and the yeast FLP/FRT systems constitute two particularly useful systems for site specific integration or excision of transgenes. In these systems, a recombinase (Cre or FLP) will interact specifically with its respective site-specific recombination sequence (lox or FRT, respectively) to invert or excise the intervening sequences. The sequence for each of these two systems is relatively short (34 bp for lox and 47 bp for FRT) and therefore, convenient for use with transformation vectors.

The FLP/FRT recombinase system has been demonstrated to function efficiently in plant cells. Experiments on the performance of the FLP/ERT system in both maize and rice protoplasts indicate that FRT site structure, and amount of the FLP protein present, affects excision activity. In general, short incomplete FRT sites leads to higher accumulation of excision products than the complete full-length FRT sites. The systems can catalyze both intra- and intermolecular reactions in maize protoplasts, indicating its utility for DNA excision as well as integration reactions. The recombination reaction is reversible and this reversibility can compromise the efficiency of the reaction in each direction. Altering the structure of the site-specific recombination sequences is one approach to remedying this situation. The site-specific recombination sequence can be mutated in a manner that the product of the recombination reaction is no longer recognized as a substrate for the reverse reaction, thereby stabilizing the integration or excision event.

In the Cre-lox system, discovered in bacteriophage P1, recombination between loxP sites occurs in the presence of the Cre recombinase (see, e.g., U.S. Pat. No. 5,658,772, specifically incorporated herein by reference in its entirety). This system has been utilized to excise a gene located between two lox sites which had been introduced into a yeast genome (Sauer, 1987). Cre was expressed from an inducible yeast GAL1 promoter and this Cre gene was located on an autonomously replicating yeast vector.

Since the lox site is an asymmetrical nucleotide sequence, lox sites on the same DNA molecule can have the same or opposite orientation with respect to each other. Recombination between lox sites in the same orientation results in a deletion of the DNA Segment located between the two lox sites and a connection between the resulting ends of the original DNA molecule. The deleted DNA segment forms a circular molecule of DNA. The original DNA molecule and the resulting circular molecule each contain a single lox site. Recombination between lox sites in opposite orientations on the same DNA molecule result in an inversion of the nucleotide sequence of the DNA segment located between the two lox sites. In addition, reciprocal exchange of DNA segments proximate to lox sites located on two different DNA molecules can occur. All of these recombination events are catalyzed by the product of the Cre coding region.

IV. General Embodiments

The methods of the present invention may be used to enhance plant crop productivity or animal survival. Both plants and animals that are raised for agricultural purposes have problems when encountering certain levels of environmental stress. By incorporating into the plant a genetic construct capable of enhancing production of stress response proteins, and then either exposing the plant to stress, or placing it in its natural environment, the stress induced proteins will be sufficient to protect the organism from deleterious or toxic effects of the environment. An animal may benefit from such an enhancement by consuming material from such a genetically altered plant.

It is an object of the present invention to provide a transgenic plant with improved tbermotolerance associated with a Hsp100 family sequence. The plant which is transgenic may be any plant species. In specific embodiments, the plant is selected from the group consisting of a cereal, grass, an ornamental plant, a crop plant, a food plant, an oil-producing plant, a synthetic product-producing plant, an environmental waste absorbing plant, a plant used for alcohol, a plant used for medicinal purposes, a plant used for recreational purposes, and a plant used for animal feed.

Cereal is herein defined as a grass which has starchy grains used for food. Examples of cereals are wheat, rye, barley, rice and oats. A grass as used herein is defined as a member of the grass family or any plant with slender leaves characteristic of the grass family. Examples of grasses are St. Augustine, hybrid Bermuda grass, rye grass is commercially available through a florist, Zoysiagrass, turfgrass and coastal Bermuda grass.

An ornamental plant is herein defined as a plant used for decorative purposes, such as is commercially available through a florist. Examples of ornamental plants include flowering plants, palms, ferns, woody plants, shrubs, ficus, evergreens and ivy.

A crop plant is herein defined as a cultivated plant and/or agricultural produce, such as grain, vegetables, legumes or fruit. Examples of crop plants include cotton, corn, sorghum, soybean, tobacco, rice, canola and mustard.

A food plant is herein defined as a plant of which part is consumed. The part for consumption may be the leaves, flowers, seeds, stems, or roots. Examples of food crops include potatoes, corn, rice, peanuts and wheat.

An oil-producing plant is herein defined as a plant of which part is utilized for oil production for consumption purposes. Examples of oil-producing plants include canola, soybean, corn, peanut, olive trees and vegetables.

A synthetic-product producing plant as used herein is defined as a plant which has been engineered to produce a synthetic product such as a plastic. For example, and as taught by Poirier et al. (1995), a plant may be altered to accumulate a plastic such as polyhydroxyalkanoates (PHAs) by expressing a nucleic acid associated with its synthesis. In an alternative embodiment, the synthetic product produced by the plant is a medicament.

An environmental waste-absorbing plant as used herein is defined as a plant which has been altered to remove environmental wastes or toxins from the environment, including soil, air or water. For example, and as taught by Bizily et al. (1999) and Bizily et al. (2000), a nucleic acid sequence is inserted into a plant genome which facilitates growth in environmentally toxic conditions and removal of the waste product or toxin present. In a specific embodiment, a bacterial nucleic acid sequence is utilized to provide such a resistance. An environmentally toxic condition is herein defined as any condition in which a pollutant, waste product, toxin, or environmentally hazardous chemical or composition is present. Examples of toxic conditions include excess mercury or unacceptable levels of radioactivity.

An alcohol plant as used herein is defined as a plant of which at least part is utilized in the production of an alcoholic beverage. Examples include grapes, hops, barley, rice, corn, grain, and wheat. Examples of alcoholic beverages include beer, wine, liquor, sake and liqueurs.

A medicinal plant as used herein is defined as a plant of which at least part is utilized for consumption or manufacture of a medicament, such as a medicine, vitamin or health-improving composition. An example is aloe vera, opium poppy (Papaver somniferum), diosgenin, derived from various species of yam (Dioscorea spp.) and used to manufacture progesterone, Echinacea, witch hazel and Ginkgo biloba. In a specific embodiment, the plant has been altered to contain a vaccine or composition capable of being consumed as part of the plant and which has prophylactic or medicinal purposes. In another embodiment, the medicinal plant is used for alleviating undesirable side effects from a separate medicine or health-improving composition, or from a medical procedure. Examples of side effects include nausea and/or vomiting, hives or pain. Another example of a medicinal plant is a nutriceutical. A nutriceutical as used herein is defined as an herb or any plant used in the treatment of disease or a medical condition. Examples of nutriceuticals include chamomile, Echinacea, garlic, gingko, ginseng, kava kava, St. John's Wort, willow bark, and tumeric.

A recreational plant as used herein is defined as a plant which is utilized for leisure, recreation, past time or other similar activities. A specific embodiment, includes a grass used for a park, golf course or a sport-playing field.

An animal feed plant as used herein is defined as a plant of which at least part is utilized in the manufacture of feed for animals. Examples of such plants are sorghum, corn and soybean. The plants may be used in a mixture of ingredients for the feed. Examples of animals which may consume such feed include cows, horses, sheep, pigs and chickens.

A skilled artisan is aware that members of the different plant categories as herein described may be present in multiple categories.

It is possible that the transgenic plant of the present invention may have other properties or characteristics which are also altered. For instance, a plant may be used which already has improved pest protection qualities or has resistance to herbicides. The improvements may be through genetic engineering or by traditional breeding practices.

Thus, in certain embodiments of the invention, transformation of a recipient cell may be carried out with more than one exogenous (selected) gene. As used herein, an “exogenous coding region” or “selected coding region” is a coding region not normally found in the host genome in an identical context. By this, it is meant that the coding region may be isolated from a different species than that of the host genome, or alternatively, isolated from the host genome, but is operably linked to one or more regulatory regions which differ from those found in the unaltered, native gene. Two or more exogenous coding regions also can be supplied in a single transformation event using either distinct transgene-encoding vectors, or using a single vector incorporating two or more coding sequences. For example, plasmids bearing the bar and aroA expression units in either convergent, divergent, or colinear orientation, are considered to be particularly useful. Further preferred combinations are those of an insect resistance gene, such as a Bt gene, along with a protease inhibitor gene such as pinII, or the use of bar in combination with either of the above genes. Of course, any two or more transgenes of any description, such as those conferring herbicide, insect, disease (viral, bacterial, fungal, nematode) or drought resistance, male sterility, drydown, standability, prolificacy, starch properties, oil quantity and quality, or those increasing yield or nutritional quality may be employed as desired.

Herbicide Resistance

The DNA segments encoding phosphinothricin acetyltransferase (bar and pat), EPSP synthase encoding genes conferring resistance to glyphosate, the glyphosate degradative enzyme gene gox encoding glyphosate oxidoreductase, deh (encoding a dehalogenase enzyme that inactivates dalapon), herbicide resistant (e.g., sulfonylurea and imidazolinone) acetolactate synthase, and bxn genes (encoding a nitrilase enzyme that degrades bromoxynil) are examples of herbicide resistant genes for use in transformation. The bar and pat genes code for an enzyme, phosphinothricin acetyltransferase (PAT), which inactivates the herbicide phosphinothricin and prevents this compound from inhibiting glutamine synthetase enzymes. The enzyme 5-enolpyruvylshikimate 3-phosphate synthase (EPSP Synthase), is normally inhibited by the herbicide N-(phosphonomethyl)glycine (glyphosate). However, genes are known that encode glyphosate-resistant EPSP synthase enzymes. These genes are particularly contemplated for use in plant transformation. The deh gene encodes the enzyme dalapon dehalogenase and confers resistance to the herbicide dalapon. The bxn gene codes for a specific nitrilase enzyme that converts bromoxynil to a non-herbicidal degradation product.

Insect Resistance

Potential insect resistance genes that can be introduced include Bacillus thuringiensis crystal toxin genes or Bt genes (Watrud et al., 1985). Bt genes may provide resistance to lepidopteran or coleopteran pests such as European Corn Borer (ECB). It is contemplated that preferred Bt genes for use in the transformation protocols disclosed herein will be those in which the coding sequence has been modified to effect increased expression in plants, and more particularly, in maize. Means for preparing synthetic genes are well known in the art and are disclosed in, for example, U.S. Pat. Nos. 5,500,365 and 5,689,052, each of the disclosures of which are specifically incorporated herein by reference in their entirety. Examples of such modified Bt toxin genes include a synthetic Bt CryIA(b) gene (Perlak et al., 1991), and the synthetic CryIA(c) gene termed 1800b (PCT Application WO 95/06128).

Protease inhibitors also may provide insect resistance (Johnson et al., 1989), and thus will have utility in plant transformation. The use of a protease inhibitor II gene, pinII, from tomato or potato is envisioned to be particularly useful. Even more advantageous is the use of a pinII gene in combination with a Bt toxin gene, the combined effect of which has been discovered to produce synergistic insecticidal activity. Other genes which encode inhibitors of the insect's digestive system, or those that encode enzymes or co-factors that facilitate the production of inhibitors, also may be useful. This group may be exemplified by oryzacystatin and amylase inhibitors such as those from wheat and barley.

Also, genes encoding lectins may confer additional or alternative insecticide properties. Lectins (originally termed phytohemagglutinins) are multivalent carbohydrate-binding proteins which have the ability to agglutinate red blood cells from a range of species. Lectins have been identified recently as insecticidal agents with activity against weevils, European Corn Borer and rootworm (Murdock et al., 1990; Czapla & Lang, 1990). Lectin genes contemplated to be useful include, for example, barley and wheat germ agglutinin (WGA) and rice lectins (Gatehouse et al., 1984), with WGA being preferred.

Genes controlling the production of large or small polypeptides active against insects when introduced into the insect pests, such as, e.g., lytic peptides, peptide hormones and toxins and venoms, form another aspect of the invention. For example, it is contemplated that the expression of juvenile hormone esterase, directed towards specific insect pests, also may result in insecticidal activity, or perhaps cause cessation of metamorphosis (Hammock et al., 1990).

Transgenic plants expressing genes which encode enzymes that affect the integrity of the insect cuticle form yet another aspect of the invention. Such genes include those encoding, e.g., chitinase, proteases, lipases and also genes for the production of nikkomycin, a compound that inhibits chitin synthesis, the introduction of any of which is contemplated to produce insect resistant plants. Genes that code for activities that affect insect molting, such as those affecting the production of ecdysteroid UDP-glucosyl transferase, also fall within the scope of the useful transgenes of the present invention.

Genes that code for enzymes that facilitate the production of compounds that reduce the nutritional quality of the host plant to insect pests also are encompassed by the present invention. It may be possible, for instance, to confer insecticidal activity on a plant by altering its sterol composition. Sterols are obtained by insects from their diet and are used for hormone synthesis and membrane stability. Therefore, alterations in plant sterol composition by expression of novel genes, e.g., those that directly promote the production of undesirable sterols or those that convert desirable sterols into undesirable forms, could have a negative effect on insect growth and/or development and hence endow the plant with insecticidal activity. Lipoxygenases are naturally occurring plant enzymes that have been shown to exhibit anti-nutritional effects on insects and to reduce the nutritional quality of their diet. Therefore, further embodiments of the invention concern transgenic plants with enhanced lipoxygenase activity which may be resistant to insect feeding.

Tripsacum dactyloides is a species of grass that is resistant to certain insects, including corn root worm. It is anticipated that genes encoding proteins that are toxic to insects or are involved in the biosynthesis of compounds toxic to insects will be isolated from Tripsacum and that these novel genes will be useful in conferring resistance to insects. It is known that the basis of insect resistance in Tripsacum is genetic, because the resistance has been transferred to Zea mays via sexual crosses (Branson and Guss, 1972). It further is anticipated that other cereal, monocot or dicot plant species may have genes encoding proteins that are toxic to insects which would be useful for producing insect resistant corn plants.

Further genes encoding proteins characterized as having potential insecticidal activity also may be used as transgenes in accordance herewith. Such genes include, for example, the cowpea trypsin inhibitor (CpTI; Hilder et al., 1987) which may be used as a rootworm deterrent; genes encoding avermectin (Campbell, 1989; Ikeda et al., 1987) which may prove particularly useful as a corn rootworm deterrent; ribosome inactivating protein genes; and even genes that regulate plant structures. Transgenic maize including anti-insect antibody genes and genes that code for enzymes that can convert a non-toxic insecticide (pro-insecticide) applied to the outside of the plant into an insecticide inside the plant also are contemplated.

Environment or Stress Resistance

Improvement of a plants ability to tolerate various environmental stresses such as, but not limited to, drought, excess moisture, chilling, freezing, high temperature, salt, and oxidative stress, also can be effected through expression of novel genes. It is proposed that benefits may be realized in terms of increased resistance to freezing temperatures through the introduction of an “antifreeze” protein such as that of the Winter Flounder (Cutler et al., 1989) or synthetic gene derivatives thereof. Improved chilling tolerance also may be conferred through increased expression of glycerol-3-phosphate acetyltransferase in chloroplasts (Wolter et al., 1992). Resistance to oxidative stress (often exacerbated by conditions such as chilling temperatures in combination with high light intensities) can be conferred by expression of superoxide dismutase (Gupta et al., 1993), and may be improved by glutathione reductase (Bowler et al., 1992). Such strategies may allow for tolerance to freezing in newly emerged fields as well as extending later maturity higher yielding varieties to earlier relative maturity zones.

It is contemplated that the expression of novel genes that favorably effect plant water content, total water potential, osmotic potential, and turgor will enhance the ability of the plant to tolerate drought. As used herein, the terms “drought resistance” and “drought tolerance” are used to refer to a plants increased resistance or tolerance to stress induced by a reduction in water availability, as compared to normal circumstances, and the ability of the plant to function and survive in lower-water environments. In this aspect of the invention it is proposed, for example, that the expression of genes encoding for the biosynthesis of osmotically-active solutes, such as polyol compounds, may impart protection against drought. Within this class are genes encoding for mannitol-L-phosphate dehydrogenase (Lee and Saier, 1982) and trehalose-6-phosphate synthase (Kaasen et al., 1992). Through the subsequent action of native phosphatases in the cell or by the introduction and coexpression of a specific phosphatase, these introduced genes will result in the accumulation of either mannitol or trehalose, respectively, both of which have been well documented as protective compounds able to mitigate the effects of stress. Mannitol accumulation in transgenic tobacco has been verified and preliminary results indicate that plants expressing high levels of this metabolite are able to tolerate an applied osmotic stress (Tarczynski et al., 1992, 1993). Altered water utilization in transgenic corn producing mannitol also has been demonstrated (U.S. Pat. No. 5,780,709).

Similarly, the efficacy of other metabolites in protecting either enzyme function (e.g., alanopine or propionic acid) or membrane integrity (e.g., alanopine) has been documented (Loomis et al., 1989), and therefore expression of genes encoding for the biosynthesis of these compounds might confer drought resistance in a manner similar to or complimentary to mannitol. Other examples of naturally occurring metabolites that are osmotically active and/or provide some direct protective effect during drought and/or desiccation include fructose, erythritol (Coxson et al., 1992), sorbitol, dulcitol (Karsten et al., 1992), glucosylglycerol (Reed et al., 1984; Erdmann et al., 1992), sucrose, stachyose (Koster and Leopold, 1988; Blackman et al., 1992), raffinose (Bernal-Lugo and Leopold, 1992), proline (Rensburg et al., 1993), glycine betaine, ononitol and pinitol (Vernon and Bohnert, 1992). Continued canopy growth and increased reproductive fitness during times of stress will be augmented by introduction and expression of genes such as those controlling the osmotically active compounds discussed above and other such compounds. Currently preferred genes which promote the synthesis of an osmotically active polyol compound are genes which encode the enzymes mannitol-1-phosphate dehydrogenase, trehalose-6-phosphate synthase and myoinositol 0-methyltransferase.

It is contemplated that the expression of specific proteins also may increase drought tolerance. Three classes of Late Embryogenic Proteins have been assigned based on structural similarities (see Dure et al., 1989). All three classes of LEAs have been demonstrated in maturing (i.e., desiccating) seeds. Within these 3 types of LEA proteins, the Type-II (dehydrin-type) have generally been implicated in drought and/or desiccation tolerance in vegetative plant parts (i.e., Mundy and Chua, 1988; Piatkowski et al., 1990; Yamaguchi-Shinozaki et al., 1992). Recently, expression of a Type-III LEA (HVA-1) in tobacco was found to influence plant height, maturity and drought tolerance (Fitzpatrick, 1993). In rice, expression of the HVA-1 gene influenced tolerance to water deficit and salinity (Xu et al., 1996). Expression of structural genes from all three LEA groups may therefore confer drought tolerance. Other types of proteins induced during water stress include thiol proteases, aldolases and transmembrane transporters (Guerrero et al., 1990), which may confer various protective and/or repair-type functions during drought stress. It also is contemplated that genes that effect lipid biosynthesis and hence membrane composition might also be useful in conferring drought resistance on the plant.

Many of these genes for improving drought resistance have complementary modes of action. Thus, it is envisaged that combinations of these genes might have additive and/or synergistic effects in improving drought resistance in crop plants such as, for example, corn. Many of these genes also improve freezing tolerance (or resistance); the physical stresses incurred during freezing and drought are similar in nature and may be mitigated in similar fashion. Benefit may be conferred via constitutive expression of these genes, but the preferred means of expressing these novel genes may be through the use of a turgor-induced promoter (such as the promoters for the turgor-induced genes described in Guerrero et al., 1990 and Shagan et al., 1993, which are incorporated herein by reference). Inducible, spatial and temporal expression patterns of these genes may enable plants to better withstand stress.

It is proposed that expression of genes that are involved with specific morphological traits that allow for increased water extractions from drying soil would be of benefit. For example, introduction and expression of genes that alter root characteristics may enhance water uptake. It also is contemplated that expression of genes that enhance reproductive fitness during times of stress would be of significant value. For example, expression of genes that improve the synchrony of pollen shed and receptiveness of the female flower parts, i.e., silks, would be of benefit. In addition it is proposed that expression of genes that minimize kernel abortion during times of stress would increase the amount of grain to be harvested and hence be of value.

Given the overall role of water in determining yield, it is contemplated that enabling corn and other crop plants to utilize water more efficiently, through the introduction and expression of novel genes, will improve overall performance even when soil water availability is not limiting. By introducing genes that improve the ability of plants to maximize water usage across a full range of stresses relating to water availability, yield stability or consistency of yield performance may be realized.

Disease Resistance

It is proposed that increased resistance to diseases may be realized through introduction of genes into plants, for example, into monocotyledonous plants such as maize. It is possible to produce resistance to diseases caused by viruses, bacteria, fungi and nematodes. It also is contemplated that control of mycotoxin producing organisms may be realized through expression of introduced genes.

Resistance to viruses may be produced through expression of novel genes. For example, it has been demonstrated that expression of a viral coat protein in a transgenic plant can impart resistance to infection of the plant by that virus and perhaps other closely related viruses (Cuozzo et al., 1988, Hemenway et al., 1988, Abel et al., 1986). It is contemplated that expression of antisense genes targeted at essential viral functions also may impart resistance to viruses. For example, an antisense gene targeted at the gene responsible for replication of viral nucleic acid may inhibit replication and lead to resistance to the virus. It is believed that interference with other viral functions through the use of antisense genes also may increase resistance to viruses. Similarly, ribozymes could be used in this context. Further, it is proposed that it may be possible to achieve resistance to viruses through other approaches, including, but not limited to the use of satellite viruses.

Increased resistance to diseases caused by bacteria and fungi also may be realized through introduction of novel genes. It is contemplated that genes encoding so-called “peptide antibiotics,” pathogenesis related (PR) proteins, toxin resistance, and proteins affecting host-pathogen interactions such as morphological characteristics will be useful. Peptide antibiotics are polypeptide sequences which are inhibitory to growth of bacteria and other microorganisms. For example, the classes of peptides referred to as cecropins and magainins inhibit growth of many species of bacteria and fungi. It is proposed that expression of PR proteins in monocotyledonous plants such as maize may be useful in conferring resistance to bacterial disease. These genes are induced following pathogen attack on a host plant and have been divided into at least five classes of proteins (Bol, Linthorst, and Comelissen, 1990). Included amongst the PR proteins are β-1, 3-glucanases, chitinases, and osmotin and other proteins that are believed to function in plant resistance to disease organisms. Other genes have been identified that have antifungal properties, e.g., UDA (stinging nettle lectin) and hevein (Broakaert et al., 1989; Barkai-Golan et al., 1978). It is known that certain plant diseases are caused by the production of phytotoxins. It is proposed that resistance to these diseases would be achieved through expression of a novel gene that encodes an enzyme capable of degrading or otherwise inactivating the phytotoxin. It also is contemplated that expression of novel genes that alter the interactions between the host plant and pathogen may be useful in reducing the ability of the disease organism to invade the tissues of the host plant, e.g., an increase in the waxiness of the leaf cuticle or other morphological characteristics.

Plant parasitic nematodes are a cause of disease in many plants, including maize. It is proposed that it would be possible to make plants resistant to these organisms through the expression of novel gene products. It is anticipated that control of nematode infestations would be accomplished by altering the ability of the nematode to recognize or attach to a host plant and/or enabling the plant to produce nematicidal compounds, including but not limited to proteins.

Mycotoxin Reduction/Elimination

Production of mycotoxins, including aflatoxin and fumonisin, by fungi associated with monocotyledonous plants such as maize is a significant factor in rendering the grain not useful. These fungal organisms do not cause disease symptoms and/or interfere with the growth of the plant, but they produce chemicals (mycotoxins) that are toxic to animals. It is contemplated that inhibition of the growth of these fungi would reduce the synthesis of these toxic substances and therefore reduce grain losses due to mycotoxin contamination. It also is proposed that it may be possible to introduce novel genes into monocotyledonous plants such as maize that would inhibit synthesis of the mycotoxin. Further, it is contemplated that expression of a novel gene which encodes an enzyme capable of rendering the mycotoxin nontoxic would be useful in order to achieve reduced mycotoxin contamination of grain. The result of any of the above mechanisms would be a reduced presence of mycotoxins on grain.

Grain Composition or Quality

Genes may be introduced into monocotyledonous plants, particularly commercially important cereals such as maize, to improve the grain for which the cereal is primarily grown. A wide range of novel transgenic plants produced in this manner may be envisioned depending on the particular end use of the grain.

The largest use of maize grain is for feed or food. Introduction of genes that alter the composition of the grain may greatly enhance the feed or food value. The primary components of maize grain are starch, protein, and oil. Each of these primary components of maize grain may be improved by altering its level or composition. Several examples may be mentioned for illustrative purposes, but in no way provide an exhaustive list of possibilities.

The protein of cereal grains including maize is suboptimal for feed and food purposes especially when fed to pigs, poultry, and humans. The protein is deficient in several amino acids that are essential in the diet of these species, requiring the addition of supplements to the grain. Limiting essential amino acids may include lysine, methionine, tryptophan, threonine, valine, arginine, and histidine. Some amino acids become limiting only after corn is supplemented with other inputs for feed formulations. For example, when corn is supplemented with soybean meal to meet lysine requirements methionine becomes limiting. The levels of these essential amino acids in seeds and grain may be elevated by mechanisms which include, but are not limited to, the introduction of genes to increase the biosynthesis of the amino acids, decrease the degradation of the amino acids, increase the storage of the amino acids in proteins, or increase transport of the amino acids to the seeds or grain.

One mechanism for increasing the biosynthesis of the amino acids is to introduce genes that deregulate the amino acid biosynthetic pathways such that the plant can no longer adequately control the levels that are produced. This may be done by deregulating or bypassing steps in the amino acid biosynthetic pathway which are normally regulated by levels of the amino acid end product of the pathway. Examples include the introduction of genes that encode deregulated versions of the enzymes aspartokinase or dihydrodipicolinic acid (DHDP)-synthase for increasing lysine and threonine production, and anthranilate synthase for increasing tryptophan production. Reduction of the catabolism of the amino acids may be accomplished by introduction of DNA sequences that reduce or eliminate the expression of genes encoding enzymes that catalyze steps in the catabolic pathways such as the enzyme lysine-ketoglutarate reductase. It is anticipated that it may be desirable to target expression of genes relating to amino acid biosynthesis to the endosperm or embryo of the seed. More preferably, the gene will be targeted to the embryo. It will also be preferable for genes encoding proteins involved in amino acid biosynthesis to target the protein to a plastid using a plastid transit peptide sequence.

The protein composition of the grain may be altered to improve the balance of amino acids in a variety of ways including elevating expression of native proteins, decreasing expression of those with poor composition, changing the composition of native proteins, or introducing genes encoding entirely new proteins possessing superior composition. Examples may include the introduction of DNA that decreases the expression of members of the zein family of storage proteins. This DNA may encode ribozymes or antisense sequences directed to impairing expression of zein proteins or expression of regulators of zein expression such as the opaque-2 gene product. It also is proposed that the protein composition of the grain may be modified through the phenomenon of co-suppression, i.e., inhibition of expression of an endogenous gene through the expression of an identical structural gene or gene fragment introduced through transformation (Goring et al., 1991). Additionally, the introduced DNA may encode enzymes which degrade zeins. The decreases in zein expression that are achieved may be accompanied by increases in proteins with more desirable amino acid composition or increases in other major seed constituents such as starch. Alternatively, a chimeric gene may be introduced that comprises a coding sequence for a native protein of adequate amino acid composition such as for one of the globulin proteins or 10 kD delta zein or 20 kD delta zein or 27 kD gamma zein of maize and a promoter or other regulatory sequence designed to elevate expression of the protein. The coding sequence of the gene may include additional or replacement codons for essential amino acids. Further, a coding sequence obtained from another species, or, a partially or completely synthetic sequence encoding a completely unique peptide sequence designed to enhance the amino acid composition of the seed may be employed. It is anticipated that it may be preferable to target expression of these transgenes encoding proteins with superior composition to the endosperm of the seed.

The introduction of genes that alter the oil content of the grain may be of value. Increases in oil content may result in increases in metabolizable-energy-content and density of the seeds for use in feed and food. The introduced genes may encode enzymes that remove or reduce rate-limitations or regulated steps in fatty acid or lipid biosynthesis. Such genes may include, but are not limited to, those that encode acetyl-CoA carboxylase, ACP-acyltransferase, β-ketoacyl-ACP synthase, plus other well known fatty acid biosynthetic activities. Other possibilities are genes that encode proteins that do not possess enzymatic activity such as acyl carrier protein. Genes may be introduced that alter the balance of fatty acids present in the oil providing a more healthful or nutritive feedstuff. The introduced DNA also may encode sequences that block expression of enzymes involved in fatty acid biosynthesis, altering the proportions of fatty acids present in the grain such as described below. Some other examples of genes specifically contemplated by the inventors for use in creating transgenic plants with altered oil composition traits include 2-acetyltransferase, oleosin, pyruvate dehydrogenase complex, acetyl CoA synthetase, ATP citrate lyase, ADP-glucose pyrophosphorylase and genes of the carnitine-CoA-acetyl-CoA shuttles. It is anticipated that expression of genes related to oil biosynthesis will be targeted to the plastid, using a plastid transit peptide sequence and preferably expressed in the seed embryo.

Genes may be introduced that enhance the nutritive value of the starch component of the grain, for example by increasing the degree of branching, resulting in improved utilization of the starch in cows by delaying its metabolism. It is anticipated that expression of genes related to starch biosynthesis will preferably be targeted to the endosperm of the seed.

Besides affecting the major constituents of the grain, genes may be introduced that affect a variety of other nutritive, processing, or other quality aspects of the grain as used for feed or food. For example, pigmentation of the grain may be increased or decreased. Enhancement and stability of yellow pigmentation is desirable in some animal feeds and may be achieved by introduction of genes that result in enhanced production of xanthophylls and carotenes by eliminating rate-limiting steps in their production. Such genes may encode altered forms of the enzymes phytoene synthase, phytoene desaturase, or lycopene synthase. Alternatively, unpigmented white corn is desirable for production of many food products and may be produced by the introduction of DNA which blocks or eliminates steps in pigment production pathways.

Most of the phosphorous content of the grain is in the form of phytate, a form of phosphate storage that is not metabolized by monogastric animals. Therefore, in order to increase the availability of seed phosphate, it is anticipated that one will desire to decrease the amount of phytate in seed and increase the amount of free phosphorous. It is anticipated that one can decrease the expression or activity of one of the enzymes involved in the synthesis of phytate. For example, suppression of expression of the gene encoding inositol phosphate synthetase (INOPS) may lead to an overall reduction in phytate accumulation. It is anticipated that antisense or sense suppression of gene expression may be used. Alternatively, one may express a gene in corn seed which will be activated, e.g., by pH, in the gastric system of a monogastric animal and will release phosphate from phytate, e.g., phytase.

Feed or food comprising primarily maize or other cereal grains possesses insufficient quantities of vitamins and must be supplemented to provide adequate nutritive value. Introduction of genes that enhance vitamin biosynthesis in seeds may be envisioned including, for example, vitamins A, E, B₁₂, choline, and the like. Maize grain also does not possess sufficient mineral content for optimal nutritive value. Genes that affect the accumulation or availability of compounds containing phosphorus, sulfur, calcium, manganese, zinc, and iron among others would be valuable. An example may be the introduction of a gene that reduced phytic acid production or encoded the enzyme phytase which enhances phytic acid breakdown. These genes would increase levels of available phosphate in the diet, reducing the need for supplementation with mineral phosphate.

Numerous other examples of improvement of maize or other cereals for feed and food purposes might be described. The improvements may not even necessarily involve the grain, but may, for example, improve the value of the corn for silage. Introduction of DNA to accomplish this might include sequences that alter lignin production such as those that result in the “brown midrib” phenotype associated with superior feed value for cattle.

In addition to direct improvements in feed or food value, genes also may be introduced which improve the processing of corn and improve the value of the products resulting from the processing. The primary method of processing corn is via wetmilling. Maize may be improved though the expression of novel genes that increase the efficiency and reduce the cost of processing such as by decreasing steeping time.

Improving the value of wetmilling products may include altering the quantity or quality of starch, oil, corn gluten meal, or the components of corn gluten feed. Elevation of starch may be achieved through the identification and elimination of rate limiting steps in starch biosynthesis or by decreasing levels of the other components of the grain resulting in proportional increases in starch. An example of the former may be the introduction of genes encoding ADP-glucose pyrophosphorylase enzymes with altered regulatory activity or which are expressed at higher level. Examples of the latter may include selective inhibitors of, for example, protein or oil biosynthesis expressed during later stages of kernel development.

The properties of starch may be beneficially altered by changing the ratio of amylose to amylopectin, the size of the starch molecules, or their branching pattern. Through these changes a broad range of properties may be modified which include, but are not limited to, changes in gelatinization temperature, heat of gelatinization, clarity of films and pastes, rheological properties, and the like. To accomplish these changes in properties, genes that encode granule-bound or soluble starch synthase activity or branching enzyme activity may be introduced alone or combination. DNA such as antisense constructs also may be used to decrease levels of endogenous activity of these enzymes. The introduced genes or constructs may possess regulatory sequences that time their expression to specific intervals in starch biosynthesis and starch granule development. Furthermore, it may be worthwhile to introduce and express genes that result in the in vivo derivatization, or other modification, of the glucose moieties of the starch molecule. The covalent attachment of any molecule may be envisioned, limited only by the existence of enzymes that catalyze the derivatizations and the accessibility of appropriate substrates in the starch granule. Examples of important derivations may include the addition of functional groups such as amines, carboxyls, or phosphate groups which provide sites for subsequent in vitro derivatizations or affect starch properties through the introduction of ionic charges. Examples of other modifications may include direct changes of the glucose units such as loss of hydroxyl groups or their oxidation to aldehyde or carboxyl groups.

Oil is another product of wetmilling of corn, the value of which may be improved by introduction and expression of genes. The quantity of oil that can be extracted by wetmilling may be elevated by approaches as described for feed and food above. Oil properties also may be altered to improve its performance in the production and use of cooking oil, shortenings, lubricants or other oil-derived products or improvement of its health attributes when used in the food-related applications. Novel fatty acids also may be synthesized which upon extraction can serve as starting materials for chemical syntheses. The changes in oil properties may be achieved by altering the type, level, or lipid arrangement of the fatty acids present in the oil. This in turn may be accomplished by the addition of genes that encode enzymes that catalyze the synthesis of novel fatty acids and the lipids possessing them or by increasing levels of native fatty acids while possibly reducing levels of precursors. Alternatively, DNA sequences may be introduced which slow or block steps in fatty acid biosynthesis resulting in the increase in precursor fatty acid intermediates. Genes that might be added include desaturases, epoxidases, hydratases, dehydratases, and other enzymes that catalyze reactions involving fatty acid intermediates. Representative examples of catalytic steps that might be blocked include the desaturations from stearic to oleic acid and oleic to linolenic acid resulting in the respective accumulations of stearic and oleic acids. Another example is the blockage of elongation steps resulting in the accumulation of C₈ to C₁₂ saturated fatty acids.

Improvements in the other major corn wetmilling products, corn gluten meal and corn gluten feed, also may be achieved by the introduction of genes to obtain novel corn plants. Representative possibilities include but are not limited to those described above for improvement of food and feed value.

In addition, it may further be considered that the corn plant be used for the production or manufacturing of useful biological compounds that were either not produced at all, or not produced at the same level, in the corn plant previously. The novel corn plants producing these compounds are made possible by the introduction and expression of genes by corn transformation methods. The vast array of possibilities include but are not limited to any biological compound which is presently produced by any organism such as proteins, nucleic acids, primary and intermediary metabolites, carbohydrate polymers, etc. The compounds may be produced by the plant, extracted upon harvest and/or processing, and used for any presently recognized useful purpose such as pharmaceuticals, fragrances, and industrial enzymes to name a few.

Further possibilities, to exemplify the range of grain traits or properties potentially encoded by introduced genes in transgenic plants, include grain with less breakage susceptibility for export purposes or larger grit size when processed by dry milling through introduction of genes that enhance γ-zein synthesis, popcorn with improved popping quality and expansion volume through genes that increase pericarp thickness, corn with whiter grain for food uses though introduction of genes that effectively block expression of enzymes involved in pigment production pathways, and improved quality of alcoholic beverages or sweet corn through introduction of genes which affect flavor such as the shrunken 1 gene (encoding sucrose synthase) or shrunken 2 gene (encoding ADPG pyrophosphorylase) for sweet corn.

Plant Agronomic Characteristics

Two of the factors determining where crop plants can be grown are the average daily temperature during the growing season and the length of time between frosts. Within the areas where it is possible to grow a particular crop, there are varying limitations on the maximal time it is allowed to grow to maturity and be harvested. For example, maize to be grown in a particular area is selected for its ability to mature and dry down to harvestable moisture content within the required period of time with maximum possible yield. Therefore, corn of varying maturities is developed for different growing locations. Apart from the need to dry down sufficiently to permit harvest, it is desirable to have maximal drying take place in the field to minimize the amount of energy required for additional drying post-harvest. Also, the more readily the grain can dry down, the more time there is available for growth and kernel fill. It is considered that genes that influence maturity and/or dry down can be identified and introduced into corn or other plants using transformation techniques to create new varieties adapted to different growing locations or the same growing location, but having improved yield to moisture ratio at harvest. Expression of genes that are involved in regulation of plant development may be especially useful, e.g., the liguleless and rough sheath genes that have been identified in corn.

It is contemplated that genes may be introduced into plants that would improve standability and other plant growth characteristics. Expression of novel genes in maize which confer stronger stalks, improved root systems, or prevent or reduce ear droppage would be of great value to the farmer. It is proposed that introduction and expression of genes that increase the total amount of photoassimilate available by, for example, increasing light distribution and/or interception would be advantageous. In addition, the expression of genes that increase the efficiency of photosynthesis and/or the leaf canopy would further increase gains in productivity. It is contemplated that expression of a phytochrome gene in corn may be advantageous. Expression of such a gene may reduce apical dominance, confer semidwarfism on a plant, and increase shade tolerance (U.S. Pat. No. 5,268,526). Such approaches would allow for increased plant populations in the field.

Delay of late season vegetative senescence would increase the flow of assimilate into the grain and thus increase yield. It is proposed that overexpression of genes within corn that are associated with “stay green” or the expression of any gene that delays senescence would be advantageous. For example, a nonyellowing mutant has been identified in Festuca pratensis (Davies et al., 1990). Expression of this gene as well as others may prevent premature breakdown of chlorophyll and thus maintain canopy function.

Nutrient Utilization

The ability to utilize available nutrients may be a limiting factor in growth of monocotyledonous plants such as maize. It is proposed that it would be possible to alter nutrient uptake, tolerate pH extremes, mobilization through the plant, storage pools, and availability for metabolic activities by the introduction of novel genes. These modifications would allow a plant such as maize to more efficiently utilize available nutrients. It is contemplated that an increase in the activity of, for example, an enzyme that is normally present in the plant and involved in nutrient utilization would increase the availability of a nutrient. An example of such an enzyme would be phytase. It further is contemplated that enhanced nitrogen utilization by a plant is desirable. Expression of a glutamate dehydrogenase gene in corn, e.g., E. coli gdhA genes, may lead to increased fixation of nitrogen in organic compounds. Furthermore, expression of gdhA in corn may lead to enhanced resistance to the herbicide glufosinate by incorporation of excess ammonia into glutamate, thereby detoxifying the ammonia. It also is contemplated that expression of a novel gene may make a nutrient source available that was previously not accessible, e.g., an enzyme that releases a component of nutrient value from a more complex molecule, perhaps a macromolecule.

Male Sterility

Male sterility is useful in the production of hybrid seed. It is proposed that male sterility may be produced through expression of novel genes. For example, it has been shown that expression of genes that encode proteins that interfere with development of the male inflorescence and/or gametophyte result in male sterility. Chimeric ribonuclease genes that express in the anthers of transgenic tobacco and oilseed rape have been demonstrated to lead to male sterility (Mariani et al., 1990).

A number of mutations have been discovered in maize that confer cytoplasmic male sterility. One mutation in particular, referred to as T cytoplasm, also correlates with sensitivity to Southern corn leaf blight. A DNA sequence, designated TURF-13 (Levings, 1990), was identified that correlates with T cytoplasm. It is proposed that it would be possible through the introduction of TURF-13 via transformation, to separate male sterility from disease sensitivity. As it is necessary to be able to restore male fertility for breeding purposes and for grain production, it is proposed that genes encoding restoration of male fertility also may be introduced.

Negative Selectable Markers

Introduction of genes encoding traits that can be selected against may be useful for eliminating undesirable linked genes. It is contemplated that when two or more genes are introduced together by cotransformation that the genes will be linked together on the host chromosome. For example, a gene encoding Bt that confers insect resistance on the plant may be introduced into a plant together with a bar gene that is useful as a selectable marker and confers resistance to the herbicide Liberty® on the plant. However, it may not be desirable to have an insect resistant plant that also is resistant to the herbicide Liberty®. It is proposed that one also could introduce an antisense bar coding region that is expressed in those tissues where one does not want expression of the bar gene product, e.g., in whole plant parts. Hence, although the bar gene is expressed and is useful as a selectable marker, it is not useful to confer herbicide resistance on the whole plant. The bar antisense gene is a negative selectable marker.

It also is contemplated that negative selection is necessary in order to screen a population of transformants for rare homologous recombinants generated through gene targeting. For example, a homologous recombinant may be identified through the inactivation of a gene that was previously expressed in that cell. The antisense construct for neomycin phosphotransferase II (NPT II) has been investigated as a negative selectable marker in tobacco (Nicotiana tabacum) and Arabidopsis thaliana (Xiang. and Guerra, 1993). In this example, both sense and antisense NPT II genes are introduced into a plant through transformation and the resultant plants are sensitive to the antibiotic kanamycin. An introduced gene that integrates into the host cell chromosome at the site of the antisense NPT II gene, and inactivates the antisense gene, will make the plant resistant to kanamycin and other aminoglycoside antibiotics. Therefore, rare, site-specific recombinants may be identified by screening for antibiotic resistance. Similarly, any gene, native to the plant or introduced through transformation, that when inactivated confers resistance to a compound, may be useful as a negative selectable marker.

It is contemplated that negative selectable markers also may be useful in other ways. One application is to construct transgenic lines in which one could select for transposition to unlinked sites. In the process of tagging it is most common for the transposable element to move to a genetically linked site on the same chromosome. A selectable marker for recovery of rare plants in which transposition has occurred to an unlinked locus would be useful. For example, the enzyme cytosine deaminase may be useful for this purpose (Stouggard, 1993). In the presence of this enzyme the compound 5-fluorocytosine is converted to 5-fluorouracil which is toxic to plant and animal cells. If a transposable element is linked to the gene for the enzyme cytosine deaminase, one may select for transposition to unlinked sites by selecting for transposition events in which the resultant plant is now resistant to 5-fluorocytosine. The parental plants and plants containing transpositions to linked sites will remain sensitive to 5-fluorocytosine. Resistance to 5-fluorocytosine is due to loss of the cytosine deaminase gene through genetic segregation of the transposable element and the cytosine deaminase gene. Other genes that encode proteins that render the plant sensitive to a certain compound will also be useful in this context. For example, T-DNA gene 2 from Agrobacterium tumefaciens encodes a protein that catalyzes the conversion of α-naphthalene acetamide (NAM) to α-naphthalene acetic acid (NAA) renders plant cells sensitive to high concentrations of NAM (Depicker et al., 1988).

It also is contemplated that negative selectable markers may be useful in the construction of transposon tagging lines. For example, by marking an autonomous transposable element such as Ac, Master Mu, or En/Spn with a negative selectable marker, one could select for transformants in which the autonomous element is not stably integrated into the genome. It is proposed that this would be desirable, for example, when transient expression of the autonomous element is desired to activate in trans the transposition of a defective transposable element, such as Ds, but stable integration of the autonomous element is not desired. The presence of the autonomous element may not be desired in order to stabilize the defective element, i.e., prevent it from further transposing. However, it is proposed that if stable integration of an autonomous transposable element is desired in a plant the presence of a negative selectable marker may make it possible to eliminate the autonomous element during the breeding process.

Non-Protein-Expressing Sequences

DNA may be introduced into plants for the purpose of expressing RNA transcripts that function to affect plant phenotype yet are not translated into protein. Two examples are antisense RNA and RNA with ribozyme activity. Both may serve possible functions in reducing or eliminating expression of native or introduced plant genes. However, as detailed below, DNA need not be expressed to effect the phenotype of a plant.

Antisense RNA

Genes may be constructed or isolated, which when transcribed, produce antisense RNA that is complementary to all or part(s) of a targeted messenger RNA(s). The antisense RNA reduces production of the polypeptide product of the messenger RNA. The polypeptide product may be any protein encoded by the plant genome. The aforementioned genes will be referred to as antisense genes. An antisense gene may thus be introduced into a plant by transformation methods to produce a novel transgenic plant with reduced expression of a selected protein of interest. For example, the protein may be an enzyme that catalyzes a reaction in the plant. Reduction of the enzyme activity may reduce or eliminate products of the reaction which include any enzymatically synthesized compound in the plant such as fatty acids, amino acids, carbohydrates, nucleic acids and the like. Alternatively, the protein may be a storage protein, such as a zein, or a structural protein, the decreased expression of which may lead to changes in seed amino acid composition or plant morphological changes respectively. The possibilities cited above are provided only by way of example and do not represent the full range of applications.

Ribozymes

Genes also may be constructed or isolated which, when transcribed, produce RNA enzymes (ribozymes) that can act as endoribonucleases and catalyze the cleavage of RNA molecules with selected sequences. The cleavage of selected messenger RNAs can result in the reduced production of their encoded polypeptide products. These genes may be used to prepare novel transgenic plants which possess them. The transgenic plants may possess reduced levels of polypeptides including, but not limited to, the polypeptides cited above.

Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes.

Several different ribozyme motifs have been described with RNA cleavage activity (Symons, 1992). Examples include sequences from the Group I self splicing introns including Tobacco Ringspot Virus (Prody et al., 1986), Avocado Sunblotch Viroid (Palukaitis et al., 1979), and Lucerne Transient Streak Virus (Forster and Symons, 1987). Sequences from these and related viruses are referred to as hammerhead ribozyme based on a predicted folded secondary structure.

Other suitable ribozymes include sequences from RNase P with RNA cleavage activity (Yuan et al., 1992, Yuan and Altman, 1994, U.S. Pat. Nos. 5,168,053 and 5,624,824), hairpin ribozyme structures (Berzal-Herranz et al., 1992; Chowrira et al., 1993) and Hepatitis Delta virus based ribozymes (U.S. Pat. No. 5,625,047). The general design and optimization of ribozyme directed RNA cleavage activity has been discussed in detail (Haseloff and Gerlach, 1988, Symons, 1992, Chowrira et al., 1994; Thompson et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozyme, the cleavage site is a dinucleotide sequence on the target RNA is a uracil (U) followed by either an adenine, cytosine or uracil (A, C or U) (Perriman et al., 1992; Thompson et al., 1995). The frequency of this dinucleotide occurring in any given RNA is statistically 3 out of 16. Therefore, for a given target messenger RNA of 1000 bases, 187 dinucleotide cleavage sites are statistically possible.

Designing and testing ribozymes for efficient cleavage of a target RNA is a process well known to those skilled in the art. Examples of scientific methods for designing and testing ribozymes are described by Chowrira et al., (1994) and Lieber and Strauss (1995), each incorporated by reference. The identification of operative and preferred sequences for use in down regulating a given gene is simply a matter of preparing and testing a given sequence, and is a routinely practiced “screening” method known to those of skill in the art.

Induction of Gene Silencing

It also is possible that genes may be introduced to produce novel transgenic plants which have reduced expression of a native gene product by the mechanism of co-suppression. It has been demonstrated in tobacco, tomato, and petunia (Goring et al., 1991; Smith et al., 1990; Napoli et al., 1990; van der Krol et al., 1990) that expression of the sense transcript of a native gene will reduce or eliminate expression of the native gene in a manner similar to that observed for antisense genes. The introduced gene may encode all or part of the targeted native protein but its translation may not be required for reduction of levels of that native protein.

A skilled artisan recognizes that the endogenous phenomenon of co-suppression in plants may be prohibitive to the generation of transgenic plants. As reviewed by Kooter et al. (1999) and Plasterk and Ketting (2000), although the exact mechanism of co-suppression is currently unknown, it appears likely that in the majority of cases it is mediated by double-stranded RNA (dsRNA) (also see Jorgensen et al. (1999)). Furthermore, a silenced transgenic graft onto a non-silenced transgenic plant triggers co-suppression in the plant, suggesting that the silencing effect can move within the plant. Also, co-suppression of an endogenous gene can occur following introduction of only a portion of the transcript, indicating that gene silencing can spread within a transgene.

Non-RNA-Expressing Sequences

DNA elements including those of transposable elements such as Ds, Ac, or Mu, may be inserted into a gene to cause mutations. These DNA elements may be inserted in order to inactivate (or activate) a gene and thereby “tag” a particular trait. In this instance the transposable element does not cause instability of the tagged mutation, because the utility of the element does not depend on its ability to move in the genome. Once a desired trait is tagged, the introduced DNA sequence may be used to clone the corresponding gene, e.g., using the introduced DNA sequence as a PCR primer together with PCR gene cloning techniques (Shapiro, 1983; Dellaporta et al., 1988). Once identified, the entire gene(s) for the particular trait, including control or regulatory regions where desired, may be isolated, cloned and manipulated as desired. The utility of DNA elements introduced into an organism for purposes of gene tagging is independent of the DNA sequence and does not depend on any biological activity of the DNA sequence, i.e., transcription into RNA or translation into protein. The sole function of the DNA element is to disrupt the DNA sequence of a gene.

It is contemplated that unexpressed DNA sequences, including novel synthetic sequences, could be introduced into cells as proprietary “labels” of those cells and plants and seeds thereof. It would not be necessary for a label DNA element to disrupt the function of a gene endogenous to the host organism, as the sole function of this DNA would be to identify the origin of the organism. For example, one could introduce a unique DNA sequence into a plant and this DNA element would identify all cells, plants, and progeny of these cells as having arisen from that labeled source. It is proposed that inclusion of label DNAs would enable one to distinguish proprietary germplasm or germplasm derived from such, from unlabelled germplasm.

Another possible element which may be introduced is a matrix attachment region element (MAR), such as the chicken lysozyme A element (Stief, 1989), which can be positioned around an expressible gene of interest to effect an increase in overall expression of the gene and diminish position dependent effects upon incorporation into the plant genome (Stief et al., 1989; Phi-Van et al., 1990).

In a specific embodiment the genetically altered plant has the antibiotic resistance gene, such as kanamycin, removed from it before its seed is planted, as taught by Meyer and colleagues. In this method homologous sequences flank the antibiotic resistance marker and therein promote homologous recombination which removes the internal antibiotic resistance marker sequences. Other methods to remove undesirable nucleic acid sequences such as antibiotic resistance markers may be utilized, such as by adding another foreign gene to the plant to express ‘helper’ proteins that induce DNA deletion, or by cross breeding plants.

The following examples are offered by way of example, and are not intended to limit the scope of the invention in any manner.

### EXAMPLE 1

### Generation of Plants with Altered Hsp101 Levels

To create transgenic Arabidopsis plants with altered levels of Hsp101 expression, the full-length cDNA sequence derived from the Columbia ecotype (Schirmer et al., 1994) was placed under the control of the constitutive CaMV 35S promoter (Koncz et al., 1992) in either the sense or antisense orientation. These constructs, or the corresponding vector without an insert, were introduced into plants by selection for the kanamycin-resistance marker on the vector. Both root tissue culture transformants of the Nössen ecotype (No-0) and vacuum infiltration transformants of the Columbia ecotype (Col-0) were obtained (Koncz et al., 1992; Bechtold and Pelletier, 1998). Independent transgenic lines were screened to evaluate Hsp101 levels by immunoblotting with an Hsp101-specific antiserum.

Among the antisense lines tested, 12 of 27 No-0 transformants had significantly reduced levels of Hsp101 expression after a mild heat stress when compared to vector controls. Surprisingly, none of the eleven Col-0 antisense plants tested exhibited a significant decrease in Hsp101 expression.

Of plants transformed with the sense construct, only one No-0 line and two Col-0 lines expressed Hsp101 constitutively. However, 17 of 25 Col-0 transformants showed significantly reduced levels of Hsp101 after heat stress, presumably as a result of co-suppression of the introduced and endogenous genes (Matzke and Matzke, 1995). In all cases, Hsp101 was undetectable in vector control transformants or wild-type plants grown at 22° C., and heat-inducible levels of Hsp101 expression were similar.

The five Nossen antisense lines (No-AS1-5) and the five Columbia co-suppression lines (Col-SUP1-5) with the greatest reductions in Hsp101 expression were propagated for further analysis. All three constitutive expression lines (No-C1, and Col-C1 and Col-C2) and several vector control lines were also propagated. Homozygous lines of each genotype were produced and No-0 plants were backcrossed twice to reduce the likelihood of propagating adventitious mutations introduced by the tissue culture transformation.

### EXAMPLE 2

### Quantification of Hsp101 Expressio

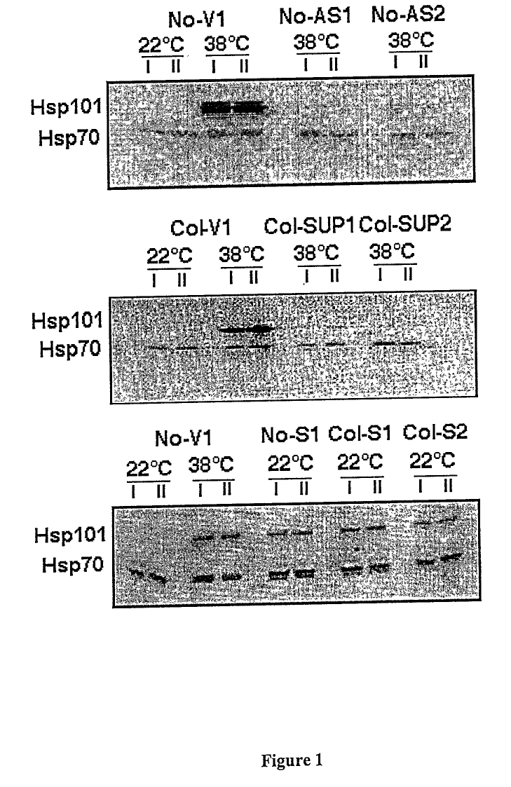

To quantify Hsp101 expression in these lines, fourteen-day-old seedlings were analyzed by protein blotting using an Hsp101 antibody and ¹²⁵I-Protein A (FIG. 1, Table 1). For FIG. 1, total cellular proteins from whole plants maintained at 22° C. or heat shocked at 38° C. for 90 min were electrophoretically separated by SDS PAGE and transferred to filters for reaction with an antiserum specific for Hsp101 and a monoclonal antibody that recognizes both constitutive and inducible members of the Hsp70 family. Immune complexes were detected with radiolabelled protein A and visualized with a phosphoimager. I and II were samples prepared from different individual plants in the same experiment.

Table 1 shows quantification of Hsp 101 expression in fourteen-day-old transgenic plants. Values of Hsp101 expression in transgenic lines after heat shock or at 22° C. were estimated using data from at least three independent experiments for each line as described in FIG. 1. Values are given relative to Hsp101 expression in vector controls after exposure to a heat treatment of 38° C. for 90 min. Experiments which were not done (n.d.) and which had no detectable immunocomplexes (undetectable) are so noted.

TABLE 1
Quantification of HSP101 Expression in
14-Day-Old Transgenic Plants.

| | | Expression after 90 min |
|---|---|---|
| Transgenic line | Expression at 22° C. | at 38° C. |
| No-AS1 | Undetectable | Undetectable- 5% |
| No-AS2 | Undetectable | Undetectable- 10% |
| No-AS3 | Undetectable | Undetectable-10% |
| No-AS4 | Undetectable | 5-10% |
| No-AS5 | Undetectable | 5-15% |
| No-AS6 | Undetectable | 50-60% |
| Col-SUP1 | Undetectable | Undetectable |
| Col-SUP2 | Undetectable | 5-10% |
| Col-SUP3 | Undetectable | 5-10% |
| Col-SUP4 | Undetectable | 10-20% |
| Col-SUP5 | Undetectable | 20-30% |
| No-C1 | 75-85% | 100-115% |
| Col-C1 | 60-65% | N.D. |
| Col-C2 | 40-50% | N.D. |

Thus, constitutive expression was assessed in plants maintained at their normal growth temperature of 22° C. Inducible expression was assessed after exposure to a standard conditioning pretreatment of 38° C. for 90 min. In wild-type plants of both ecotypes this heat treatment strongly induces Hsp101, together with other HSPs (Osteryoung et al., 1993; Schirmer et al., 1994; Wehmeyer et al.,1996), and induces tolerance to more severe heat shocks. The pretreatment itself did not reduce viability in any of the lines. To control for variations in protein loading, blots were also reacted with antibody 7.10 (Velazquez et al., 1983), which recognizes both constitutive and heat-inducible members of the Hsp70 family. (These ˜70 kDa proteins comigrate on the gels; all control samples should have the same level of expression and all heat-shocked samples should have an approximately 2-3-fold higher level.) Immunoblotting of the same samples with antibodies to the cytosolic class I small HSPs (Wehmeyer et al., 1996) demonstrated a normal heat-shock response in all plants. Changes in Hsp101 levels did not affect the expression of other proteins as far as could be detected by Coomassie.

All vector control plants strongly expressed Hsp101 after the 38° C. treatment. In the fourteen-day-old seedlings of antisense lines, Hsp101 expression was severely reduced. In many cases it was undetectable, in others it was 5%-10% of the levels observed in vector controls. In one co-suppression line, Hsp101 was so profoundly reduced that it was undetectable; in others expression was 5-30% that of vector controls (Table 1).

As expected, in wild-type plants, vector controls, antisense and co-suppression lines, Hsp101 was not detectable at normal growth temperatures (22° C.). The three constitutive lines expressed Hsp101 at high levels, close to those observed in wild-type and vector controls only after a full tolerance-inducing heat treatment at 38° C. for 90 min.

### EXAMPLE 3

### Altered Hsp101 Expression Does not Affect Growth in the Absence of Severe Heat Stress

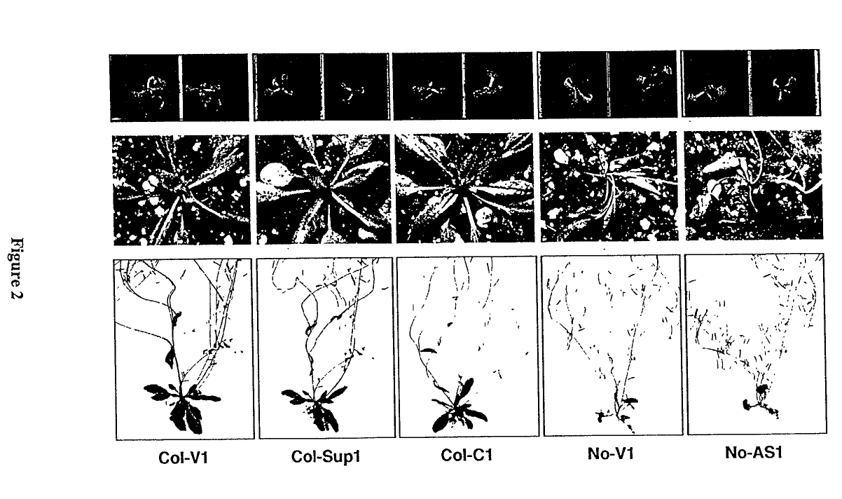

The selected transgenic lines were first analyzed for general growth phenotypes at different life stages. Neither reduced nor constitutive Hsp101 expression caused any obvious phenotype (FIG. 2). The absence of a detectable detrimental effect in the presence of constitutive Hsp101 expression, particularly while achieving a significant benefit regarding stress tolerance (see Examples below), is significant in a cell which already expresses a multitude of heat shock-related genes. It should be noted that there are ecotype-specific morphological differences between Col-0 and No-0 lines, but no changes associated with Hsp101 transgenes.

Germination times and rates, growth rates, time to flowering and seed yields were all comparable to plants transformed with the vector alone. Moreover, no differences were observed when antisense plants and control plants were grown to flowering under continuous mild heat stress (at 30° C.). Thus, wild-type levels of Hsp101 are not required for growth at normal or moderately elevated temperatures, nor does constitutive expression of the protein cause any noticeable harm.

### EXAMPLE 4

### Hsp101 is Essential for Induced Thermotolerance

To determine if Hsp101 plays a role in induced thermotolerance, vector controls and plants with reduced levels of Hsp101 were analyzed in assays involving pretreatment, severe heat stress, or combinations thereof. In these, and all other experiments presented herein, plants with altered Hsp101 levels were grown and heat treated on the same plates as vector control plants to reduce other sources of variation.

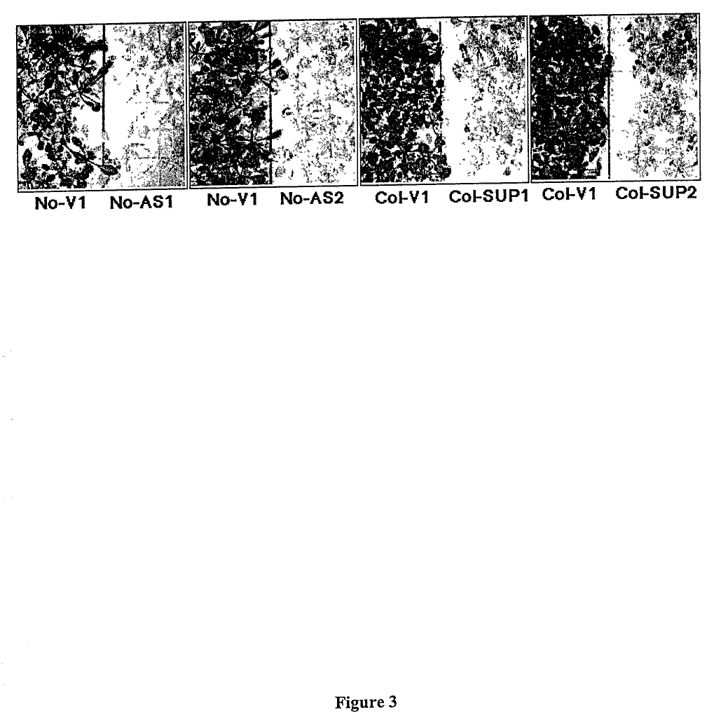

Plants were grown on defined medium (GM plates) for fourteen days and then subjected to a 45° C. heat shock for 2 hr, with or without a conditioning pretreatment at 38° C. for 90 min (FIG. 3). Plants were then returned to 22° C. Their viability was assessed daily and photographically recorded on the fifth day after stress. Two vector control lines were tested from each ecotype, five No-0 antisense lines, and five Col-0 co-suppression lines.

Plants of all genotypes died within three days when they were exposed directly to 45° C. As with wild-type plants, conditioning allowed vector controls of both the No-0 and the Col-0 ecotypes to survive the otherwise lethal heat stress (FIG. 3). Representative plates containing plants from vector control lines (No-V1 or Co-V1) and two antisense (No-AS1 or No-AS2) or co-suppression lines (Col-SUP1 or Col-SUP2) were photographed 5 days after return to 22° C.

Although vector control plants exhibited some growth delay after heat shock, after five days of recovery at 22° C., virtually all plants were green and healthy. Immediately after heat shock, No-0 antisense plants and Col-0 co-suppression plants appeared identical to the vector controls. However, in the ensuing days of recovery at 22° C., they did not continue growing and most plants died (FIG. 3). In lines with the most severe reductions in Hsp101 expression, all plants died within five to six days; thus, they exhibited some residual tolerance compared to unconditioned plants, which died within three days after heat shock.

The ability to recover from heat stress correlated with the quantity of Hsp101 produced. Lines with higher residual Hsp101 expression (e.g. Col-SUP2), recovered from heat stress somewhat better than lines with lower levels (e.g. No-AS1) (Table 1). That is, a larger fraction of plants with detectable Hsp101 expression retained some green tissue five days after heat stress. In some experiments, a few plants with higher levels of Hsp101, lines No-AS5, Col-SUP4 and Col-SUP5, survived beyond five days (data not shown). However, even these lines exhibited much more damage than vector controls.

### EXAMPLE 5

### Hsp101 is Required for Basal Thermotolerance During Germination

In addition to being induced by heat stress, Hsp101 is subject to developmental regulation. The protein accumulates to a high level during the course of seed formation at normal temperatures, remains present in mature seeds and disappears within a few days of germination. These observations prompted examination of basal thermotolerance during early development and the role of Hsp101.

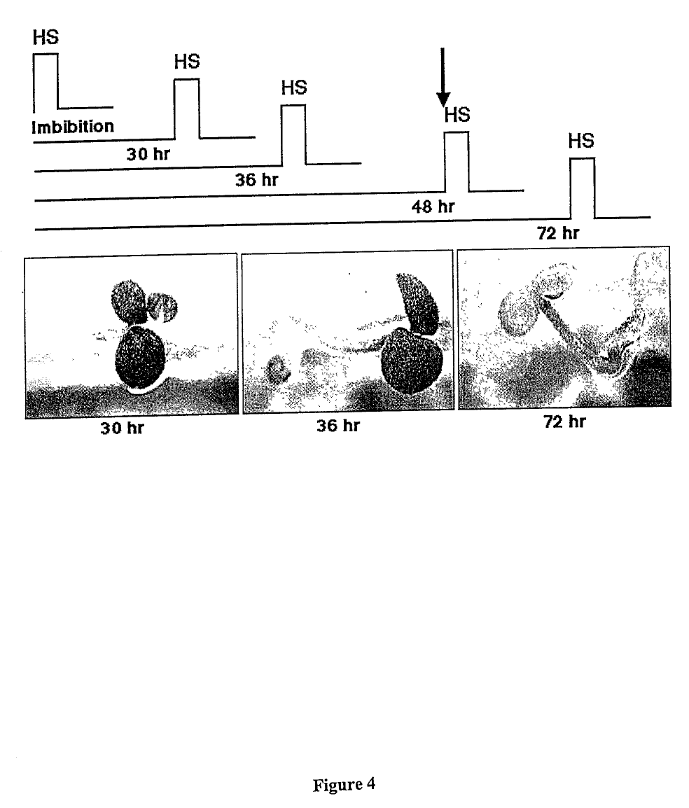

First, basal thermotolerance was examined in seeds from vector control lines. Seeds were plated on medium and heat-shocked at 47° C. for 2 hr after various periods at 22° C. Germinating seeds exhibited a remarkable ability to recover from the detrimental effects of this severe heat shock. Although development was delayed by five to seven days compared to unstressed controls, virtually all seeds heat shocked either 30 min or 30 hr after plating eventually produced healthy plants (FIG. 4). Representative plants were photographed five days after heat stress. As indicated by the arrow, all seedlings heat shocked after 48 hr of germination died.

In the next 18 hr, as the levels of Hsp101 declined thermotolerance was lost. Most germinating seeds heat shocked after 36 hr of development recovered, but survival rates were slightly lower than at 30 hr. None of the seedlings heat shocked after 48 hr of imbibition were able to survive the 47° C. heat shock (FIG. 4).

To test directly the role of Hsp101 in this high level of thermotolerance during and after germination, seeds of antisense and co-suppression lines were examined together with vector controls. Seeds (10 mg) of each genotype were used to prepare protein samples and proteins were separated on SDS-PAGE. Lanes for AS4 and AS5 were slightly underloaded, as confirmed in other experiments. Immunocomplexes were visualized by ECL. Seeds (10 mg) of each genotype were used to prepare protein samples and proteins were separated on SDS-PAGE. Lanes for AS4 and AS5 were slightly underloaded, as confirmed in other experiments. Immunocomplexes were visualized by ECL.

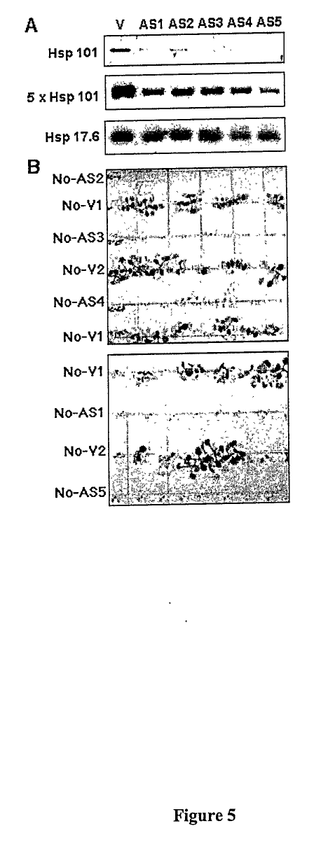

First, levels of Hsp101 expression were determined. All of the antisense lines showed strongly reduced expression of Hsp101 in both mature (dry) and germinating seeds (FIG. 5A). The decreased Hsp101 expression did not appear to affect levels of class 1 small HSPs which are also present in seeds (Wehmeyer et al.,1996). Surprisingly, in mature and germinating seeds of the co-suppression lines Hsp101 was expressed at nearly the same levels as in wild-type. Seeds (10 mg) of each genotype were used to prepare protein samples and proteins were separated on SDS-PAGE. Lanes for AS4 and AS5 were slightly underloaded, as confinned in other experiments. Immunocomplexes were visualized by ECL.

Next, seeds from antisense lines, co-suppression lines, and vector control lines were exposed to 47° C. for 2 hr immediately after seed plating, or after 30 hr, 36 hr, 48 hr and 72 hr of germination. The majority of germinating vector control and co-suppression seeds continued to develop after the heat shock at the first three time points and eventually produced healthy plants (FIG. 5B). Germinating antisense seeds failed to develop in all cases (FIG. 5B) (Representative plates were photographed ten days after heat shock).

A close examination of antisense seeds heat stressed after 36 hr of imbibition showed that the radicle emerged in some cases, indicating that elongation continued for some time. However, seedlings then stopped growing and died. Thus, reduced levels of Hsp101 did not cause immediate lethality, but rather failure to recover from heat shock.

### EXAMPLE 6

### Constitutive Hsp101 Expression Provides an Advantage to Plants Heat Shocked without Conditioning

The above experiments demonstrate that Hsp101 is required both for induced thermotolerance and for the naturally high levels of basal thermotolerance observed in germinating seedlings. However, there are likely to be many factors involved in stress tolerance, and it does not necessarily follow that overexpression of Hsp101 alone would be able to provide tolerance to otherwise sensitive plants. To investigate this possibility, plants were examined that constitutively express Hsp101 at normal temperatures in the absence of a conditioning pretreatment. As demonstrated above, fourteen-day-old plants were extremely sensitive to high temperatures if they were not given a conditioning pretreatment. When plants of this age from all three constitutive expression lines (No-C1, Col-C1, Col-C2) were exposed to the standard killing heat shock (45° C. for 2 hr) they also died. Thus, constitutive expression of Hsp101 alone at a moderate level did not provide the remarkable degree of thernotolerance that is conferred by a conditioning heat pretreatment.

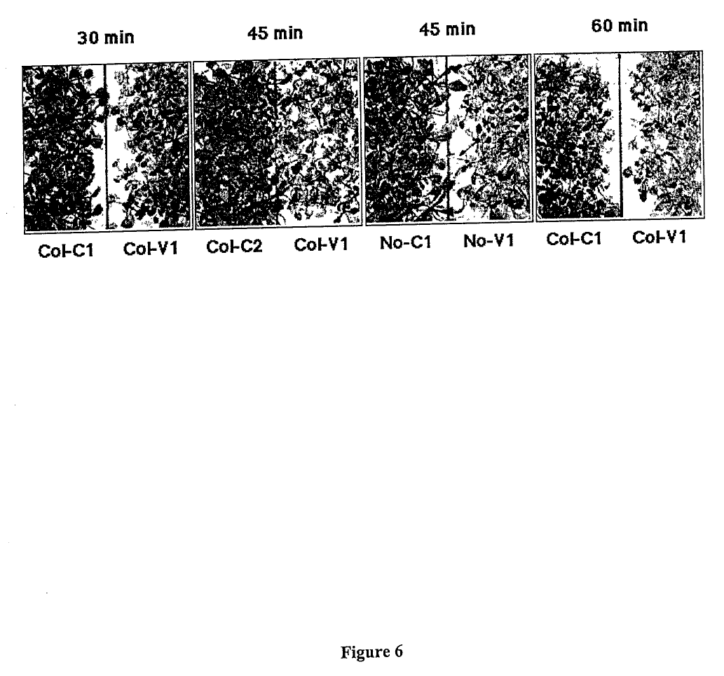

To determine if Hsp101 might provide a survival advantage under less severe conditions, fourteen-day-old seedlings were given shorter heat shocks at 45° C. and their viability was assessed daily over the following ten days. In this case, all three constitutive expression lines, No-C1, Col-C1, and Col-C2, showed a very significant advantage in comparison to vector controls (Table 2 representative examples FIG. 6B). Representative plates containing vector control (No-V1, Col-V1) and constitutive expression plants (No-C1, Col-C1 and Col-C2) were photographed five days after return to 22° C.

With a short heat shock of 15 min, all plants looked as healthy as unstressed plants and there were no distinctions between lines even after five days of recovery (Table 2). Table 2 shows that there is a growth advantage of heat shocked plants that constitutively express Hsp101. Plants from several experiments, such those shown in FIG. 5, were scored on day five. Scoring notations are as follows: ++++, plants appear as healthy as unheated controls; +++, nearly as healthy as unheated controls with some yellow tissue evident, ++, most plants have some bleached and withered leaves, all exhibited developmental delay, some individual plants dead; +/− most plants die, green tissue still evident on many plants after 5 days;− all plants dead within five days, patches of green tissue present on only a few.

With exposures of 30 min, no differences were apparent between the constitutive lines and the vector controls immediately after heat shock (Table 2 and FIG. 6). However, in the days following, vector controls bore obvious signs of stress: most plants had some bleached and withered leaves and some individual plants died. In contrast, plants from all three constitutive lines appeared as healthy as unstressed plants of the same age.

With plants given a 45 min heat shock, most of the vector controls died during the subsequent five to six days, while most of the constitutive expression plants survived. After exposure to 45° C. for 60 min the No-C1 and Col-C1 plants displayed withered leaves and were developmentally delayed. However, most of the No-C1and Col-C1 plants survived. By day ten, they were noticeably more vigorous and had clearly returned to normal growth. All of the vector control plants, however, had died. The line with the lowest level of constitutive expression of Hsp101, Col-C2, did not recover from the 60 min heat shock as well as the No-C1 and Col-C1 lines. However, even plants of this line were much less affected than vector controls (Table 2). By day six a larger fraction of plant tissue was green; and by day ten a significant fraction of plants survived and had returned to growth.

TABLE 2
Growth Advantage of Heat Shocked Plants
Constitutively Expressive HSP101.

Survival after a Period at 45° C.

| | 15 min | 30 min | 45 min | 60 min |
|---|---|---|---|---|
| Vector | + + + + | + + | ± | − |
| No-C1 | + + + + | + + + | + + + | + + |
| Col-C1 | + + + + | + + + | + + + | + + |
| Col-C2 | + + + + | + + + | + + + | ± |

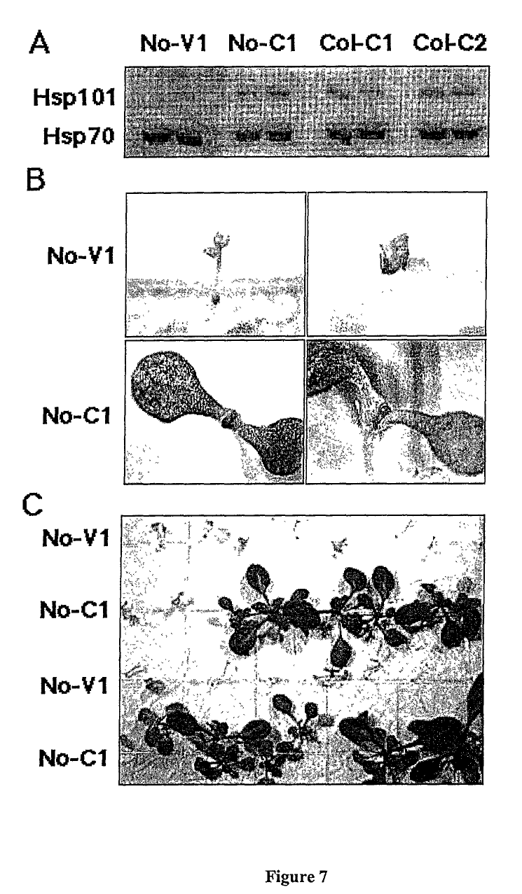

Plants from several experiments, such those shown in FIG. 7, were scored on day 6. ++++, plants appear as healthy as unheated controls; +++, plants appear almost as healthy as unheated as unheated controls, with some yellow tissue evident; ++ most plants have some bleached and withered leaves, all exhibited developmental delay, and some individual plants died; ±, most plants died, green tissue was still evident on many plants after 6 days, −, all plants died within 6 days, patches of green tissue were visible on only a few.

The effects of constitutive Hsp101 expression on newly germinated seedlings was also examined. In contrast to wild-type and vector control lines, three-day-old seedlings of all constitutive lines contained significant amounts of Hsp101 protein (FIG. 7A). Total proteins from pooled seedlings grown at 22° C. were analyzed as in FIG. 1. Constitutive expression of Hsp101 at day three was not as high relative to Hsp70 as in fourteen-day-old plants of the same genotype (see FIG. 1). Vector controls did not contain Hsp101 at this developmental stage.

When three-day-old seedlings of all genotypes were exposed to 47° C. for 2 hr none survived. However, with less severe heat shocks (47° C. for 30 min or 45 min) very strong differences in survival appeared between constitutive and vector control lines. Similar results were obtained for all three constitutive lines. Representative data for the No-C1 line and one vector control are shown in FIG. 7B and C. Representative seedlings of vector control (No-V1) and constitutive line No-C1 are shown two days after exposure to heat shock at the same magnification (Olympus DF plan 1×).

Two days after a 30 min heat shock at 47° C., stress-related damage was seen in both vector control and constitutive expression seedlings. However, most seedlings from constitutive lines were much further developed. They were displaying their first pair of adult leaves and expanded cotyledons. Vector control seedlings had no adult leaves but only small cotyledons with bleached patches. (The four photographs in FIG. 7B were taken at the same magnification.) Two weeks after the heat shock, these early signs of recovery had translated into vigorous growth for most constitutive expression plants, while vector controls had grown little if at all (FIG. 7C). Thus, the loss of basal thermotolerance observed in early development, as seedlings lose their store of Hsp101, can be partially reversed by constitutive expression of Hsp101.

### EXAMPLE 7

### An Increase in Hsp101 Quantity is Related to an Increase in Seed Quality

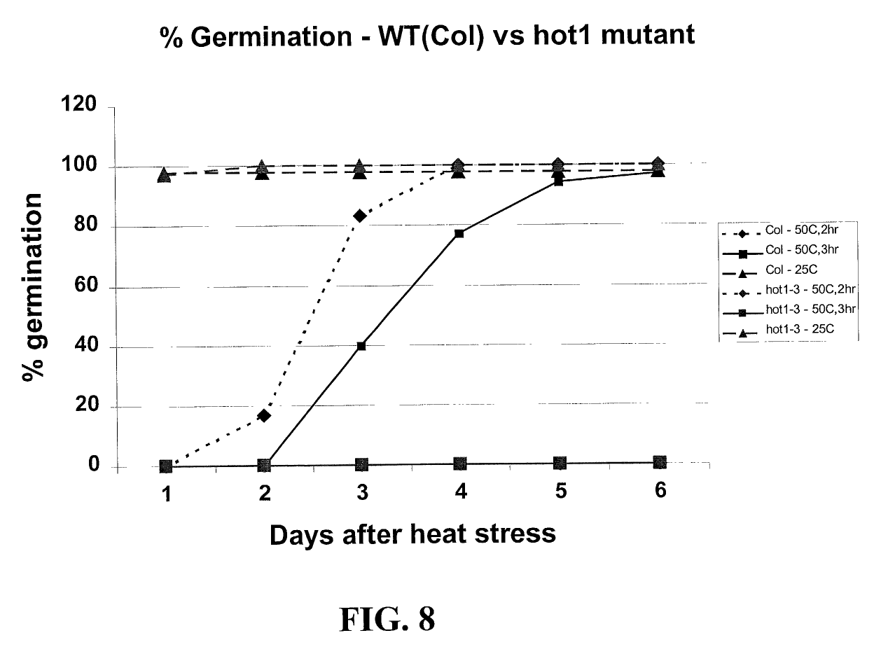

Effects on seed tolerance were tested in seeds from an Arabidopsis insertional mutant of Hsp101 that fails to express any Hsp101 protein. In this regard it is similar to the antisense plants described herein, but a skilled artisan recognizes the phenotype and genotype are much more strict because there can be no leakage. FIG. 8 illustrates the percentage of Arabidopsis seed germination after heat treatment of wild type (Col) vs. the Hsp101 mutant.

Seeds of wild type or the Hsp101 mutant were placed on filter paper disks soaked in water and incubated in the light for 18 hrs at 25° C. Seeds were then either kept at 25° C. in the light or placed for 2 or 3 hrs directly at 50° C. After treatment seeds were returned to the light at 25° C. and the number of germinated seeds were counted each 24 hrs following the heat treatment. Note that none of the heat treated Hsp101 mutant seeds germinated. The line for the 2 hr, 50° C. treatment of the mutant lies directly under the 3 hr, 50° C. treatment so it is not visible. In the absence of heat stress 25° C. sample, Hsp101 mutant seeds show normal germination.

The heat sensitivity of Hsp101 mutant seeds is a measure of seed quality and/or is a measure of the ability to germinate and establish vigorous seedlings under less than optimal conditions or after long storage. Thus, in a specific embodiment of the present inveniton, an increase in seed content of Hsp101 increases seed quality. A skilled artisan recognizes that this method, as enabled by the teachings provided herein, may be extrapolated to other plant species.

### EXAMPLE 8

### Heavy Metal Tolerance in Transgenic Hsp101 Plants

It is known that Hsp101 in yeast is induced by heavy metals. Therefore, a similar effect was tested in Arabidopsis plants. The plants were treated with 1 mM of cadmium sulfate. Plants were grown for 10 days on plates with 10 ml mineral agar medium and then flooded with 10 ml of 2 mM of Cd solution to make final concentration 1 mM. Plants were sampled for western analysis at different time points—2, 6, 12 and 24 hr after treatment, and Hsp101 accumulation was observed. Accumulation was not as high as seen following a 38° C. heat stress. A skilled artisan is aware of other examples of heavy metals, including silver, palladium, rhodium, platinum, gold, and mercury.

### EXAMPLE 9

### Hsp101 has a Direct Role in Thermotolerance of Plants

The experiments demonstrated herein show that the expression of a specific HSP plays a crucial role in the thermotolerance of a plant. Numerous studies from other laboratories have previously documented a correlation between HSP inductions and adaptation to stress in plants (Howarth and Skot, 1994; Lee et al., 1995; Lee and Schöffl, 1996; Nover, 1990; Prändl et al., 1998; Vierling, 1991; Yeh et al., 1994), but these experiments did not address the question of which HSPs might play a crucial role. Indeed, because other physiological changes generally occurred in the same plants it could not be determined whether HSP induction served vital or peripheral functions. The role of Hsp101 is herein established by several mutually supportive arguments.

First, alterations in thermotolerance were linked to alterations in heat tolerance by three different types of genetic manipulation: inhibiting Hsp101 expression through the production of antisense RNAs or by co-suppression impaired thermotolerance, whereas over-expressing Hsp101 enhanced it. Second, in each case multiple independent transformants that affected Hsp101 in the same manner displayed the same change in thermotolerance and no transformants that substantially affected Hsp101 expression failed to affect thermotolerance. Third, in experiments where conditions were sensitive enough to detect them, dosage relationships were apparent. Constitutive lines with the highest levels of Hsp101 expression were the best able to withstand heat stress and antisense lines with the strongest inhibition of Hsp101 expression were the most severely affected by heat stress. Fourth, changes in Hsp101 expression altered both acquired and basal thermotolerance. Finally, when the effects of antisense and co-suppression on Hsp101 expression diverged, their effects on tolerance also diverged: both impaired Hsp101 expression and both impaired thernotolerance in fourteen-day-old seedlings; only antisense expression reduced the developmentally regulated induction of Hsp101 in seeds and only antisense reduced thermotolerance during seed germination.

These experiments were prompted by the identification of Arabidopsis Hsp101 as a protein that is strongly induced by heat, homologous to the well-studied yeast protein Hsp104, and able to partially compensate for the loss of thermotolerance caused by hsp104 deletions in yeast (Schirmer et al., 1994). Even so, the remarkably similar effects of Hsp104 in a simple microbe and Hsp101 in a complex vascular plant are surprising. Yeast cells have multiple strategies for surviving stress (Eleutherio et al., 1993; Ruis and Schuller, 1995;; Zahringer et al., 1998; Singer and Lindquist, 1997; Moskvina et al., 1998; Simon et al., 1999) and it can only be expected that plants will have at least as many (Bohnert et al., 1995; Smimoff, 1998). Moreover, plants typically have numerous redundant and closely related genes. Indeed, several other members of the Hsp100 family, including other stress-inducible members are present in Arabidopsis (Elizabeth Vierling, unpublished results; Nakashima et al., 1997; Nielsen et al., 1997; Shanklin et al., 1995; Weaver et al., 1999). Yet this single protein plays such a pivotal role that in both organisms 1) inhibiting its expression during conditioning pretreatments had disastrous effects on the induction of thermotolerance; 2) inhibiting its developmentally regulated induction (in yeast stationary phase cells and spores (Sanchez et al., 1992), in plant seeds) severely reduces the high basal thermotolerance that characterizes these stages of development; 3) the protein appears to be less crucial in preventing stress damage than in allowing recovery from it; 4) changing the levels of Hsp101 (unlike many other HSPs and tolerance factors) had little effect on normal growth and development, and 5) expressing the protein at times when it would normally not be expressed was sufficient to confer higher basal levels of thermotolerance.

Having established that Hsp101 plays a major role in thermotolerance, it is of significant interest to understand the mechanism by which the protein functions and to define the targets that are protected. Evidence from Saccharomyces cerevisiae suggests that Hsp104 acts in vivo to reactivate proteins aggregated by high temperatures (Parsell et al., 1994b). In addition, the ability to reactivate denatured proteins has been demonstrated in vitro with purified Hsp104 and chemically denatured substrates. To reactivate proteins in vitro, Hsp104 requires the assistance of Hsp40 and Hsp70 (Glover and Lindquist, 1998). These data support a model in which Hsp104 performs the first step in dissociating protein aggregates so that Hsp70 and Hsp40 can recognize the denatured substrate and complete the refolding process. Consistent with these data, bacterial homologs of Hsp104, which are also required for stress tolerance, have recently been shown to have the same capacity to disaggregate proteins in vitro in cooperation with bacterial Hsp70 and Hsp40 homologs (Motohashi et al., 1999; Zolkiewski, 1999; Mogk et al., in press). The function of Hsp104 in protein disaggregation proteins also parallels the defined activities of other proteins belonging to a larger related family of ATPases, the AAA+ ATPases, many of which act to alter the oligomeric state of other protein complexes. Given the high sequence similarity of Hsp101 with yeast Hsp104, and their conserved functions in thermotolerance, in a specific embodiment Hsp101 in Arabidopsis is also acting to facilitate reactivation of proteins denatured by heat.

Another, though not necessarily mutually exclusive, activity has been suggested for Hsp101 by Gallie and colleagues (Wells et al., 1998). They reported that Hsp101 from tobacco and wheat positively regulates the translation of tobacco mosaic RNA through direct interaction with the sequence in the viral 5′ leader. Since Hsp101 is strongly expressed in seedlings and mature plants following heat stress, this might represent a specific mechanism for plant viruses to regulate their replication and mobility in response to the health of their host and/or a mechanism for taking advantage of the host stress response upon infection. Alternatively, or in addition, Hsp101 could affect the translation of some cellular mRNAs, and thereby contribute to thermotolerance. However, 5′ leader sequences to which Hsp101 binds have not been identified in cellular mRNAs. Also, the significant decrease of Hsp101 levels in the antisense and co-suppression lines did not lead to any noticable changes in expression of other proteins, including other HSPs, which might be the logical targets for translational enhancement during heat stress.

The finding that Hsp101 plays a crucial role in thermotolerance in plants together with the conserved function of Hsp101, suggest that engineering plants to express increased Hsp101 levels may improve survival during periods of acute environmental stress. In this regard it is important that the level of constitutive Hsp101 expression achieved increases heat tolerance without compromising growth at normal temperatures. This contrasts with other efforts to engineer stress tolerance in plants, which in many cases, such as constitutive expression of the multiple stress-response transcription factor DREB1A or a subunit of trehalose synthase (TPS1) (Holmstrom et al., 1996; Kasuga et al., 1999; Smirnoff and Bryant, 1999), produce disadvantageous growth phenotypes. With inducible promoters that might produce even higher levels of Hsp101 accumulation, much higher levels of heat tolerance might be attainable, as has already been achieved with yeast (Lindquist and Kim, 1996). Manipulation of Hsp101 expression therefore holds considerable promise in protecting plants at many life stages from irreversible stress-induced damage.

### EXAMPLE 10

### Methods for Vector Construction and Plant Transformation

The EcoRI insert of pBSKHsp100 containing the full-length cDNA of Columbia Hsp101 (Schirmer et al., 1994) was cloned into the EcoRI site of pBICaMV. Sense and antisense constructs were identified by restriction analysis and subsequently sequenced.

Plasmid DNA for sense, antisense vector, or vector without insert were transformed into Agrobacteria strain LB4400 for tissue culture transformation and Agrobacteria strain GV3101 for vacuum infiltration (Koncz et al., 1992). DNA of three independent transformants was isolated, transformed in E. coli (DH5α), and plasmid DNA was prepared for restriction analysis to confirm the presence of the respective construct in the Agrobacteria.

Root tissue culture transformation with No-0 plants was performed as described (Koncz et al., 1992). For vacuum infiltration with Col-0 plants, was followed a modified version of the protocol by Bechtold and Pelletier (Bechtold and Pelletier, 1998).

A skilled artisan recognizes several methods of transformation may be employed to introduce the genetic construct into a plant cell. For plants that are relatively easy to transform, for example, tobacco, brassica (canola oil production), potatoes, tomatoes, and the like, the HSP104 gene can be inserted into an Agrobacteria vector which is employed for a standard transformation procedure into a host plant cell.

For corn, rice and other species generally more difficult to transform, a particle accelerator gun is the currently preferred method for transformation. For this method, solutions of Hsp100 family nucleic acid are coated onto tungsten pellets and embryonic or pollen tissue are bombarded. The successful transformations may be selected by co-expression of a selectable gene included in the nucleic acid used for transformation.

It is contemplated that additional copies of the wild-type plant gene or a functional equivalent from another organism will increase the ability of plants to tolerate heat, desiccation, and other stresses. In some cases, tolerance might be achieved with smaller modules of the gene product. These might even be assembled separately and brought together only in the final composition. For example, the two nucleotide binding domains which contribute to Hsp100 function may be brought into the cells on separate vectors and the proteins themselves may directly co-assemble into a functional unit. It is also envisioned that the coding sequences for the stress protective protein may be placed under a variety of other regulatory systems so that they would be expressed only at particular times in development or in particular cells or tissues.

Suitable methods for plant transformation for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety), by Agrobacterium-mediated transformation (U.S. Pat. Nos. 5,591,616 and 5,563,055; both specifically incorporated herein by reference) and by acceleration of DNA coated particles (U.S. Pat. Nos. 5,550,318; 5,538,877; and 5,538,880; each specifically incorporated herein by reference in its entirety), etc. Through the application of techniques such as these, maize cells as well as those of virtually any other plant species may be stably transformed, and these cells developed into transgenic plants. In certain embodiments, acceleration methods are preferred and include, for example, microprojectile bombardment and the like.

Electroporation

Where one wishes to introduce DNA by means of electroporation, it is contemplated that the method of Krzyzek et al. U.S. Pat. No. 5,384,253, incorporated herein by reference in its entirety) will be particularly advantageous. In this method, certain cell wall-degrading enzymes, such as pectin-degrading enzymes, are employed to render the target recipient cells more susceptible to transformation by electroporation than untreated cells. Alternatively, recipient cells are made more susceptible to transformation by mechanical wounding.

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plants (Bates, 1994; Lazzeri, 1995). For example, the generation of transgenic soybean plants by electroporation of cotyledon-derived protoplasts is described by Dhir and Widholm in Intl. Patent Appl. Publ. No. WO 9217598 (specifically incorporated herein by reference). Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

Microprojectile Bombardment

A preferred method for delivering transforming DNA segments to plant cells in accordance with the invention is microprojectile bombardment (U.S. Pat. Nos. 5,550,318; 5,538,880; 5,610,042; and PCT Application WO 94/09699; each of which is specifically incorporated herein by reference in its entirety). In this method, particles may be coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

Microprojectile bombardment techniques are widely applicable, and may be used to transform virtually any plant species. Examples of species for which have been transformed by microprojectile bombardment include monocot species such as maize (PCT Application WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casas et al., 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al. 1994), peanut (Singsit et al., 1997), cotton (McCabe and Martinell, 1993), tomato (VanEck et al. 1995), and legumes in general (U.S. Pat. No. 5,563,055, specifically incorporated herein by reference in its entirety).

Agrobacterium-mediated Transformation

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety.

Agrobacterium-mediated transformation is most efficient in dicotyledonous plants and is the preferable method for transformation of dicots, including Arabidopsis, tobacco, tomato, and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., 1997; Zhang et al., 1997; U.S. Pat. No. 5,591,616, specifically incorporated herein by reference in its entirety), wheat (McCormac et al., 1998), barley (Tingay et al., 1997; McCormac et al., 1998), and maize (Ishidia et al., 1996).

Modem Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described (Rogers et al., 1987) have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

Other Transformation Methods

Transformation of plant protoplasts can be achieved using methods based on calcium phosphate precipitation, polyethylene glycol treatment, electroporation, and combinations of these treatments (see, e.g., Potrykus et al., 1985; Lorz et al., 1985; Omirulleh et al., 1993; Fromm et al., 1986; Uchimiya et al., 1986; Callis et al., 1987; Marcotte et al., 1988).

Application of these systems to different plant strains depends upon the ability to regenerate that particular plant strain from protoplasts. Illustrative methods for the regeneration of cereals from protoplasts have been described (Fujimara et al., 1985; Toriyama et al., 1986; Yamada et al., 1986; Abdullah et al., 1986; Omirulleh et al., 1993 and U.S. Pat. No. 5,508,184; each specifically incorporated herein by reference in its entirety). Examples of the use of direct uptake transformation of cereal protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

To transform plant strains that cannot be successfully regenerated from protoplasts, other ways to introduce DNA into intact cells or tissues can be utilized. For example, regeneration of cereals from immature embryos or explants can be effected as described (Vasil, 1989). Also, silicon carbide fiber-mediated transformation may be used with or without protoplasting (Kaeppler, 1990; Kaeppler et al., 1992; U.S. Pat. No. 5,563,055, specifically incorporated herein by reference in its entirety). Transformation with this technique is accomplished by agitating silicon carbide fibers together with cells in a DNA solution. DNA passively enters as the cell are punctured. This technique has been used successfully with, for example, the monocot cereals maize (PCT Application WO 95/06128, specifically incorporated herein by reference in its entirety; Thompson, 1995) and rice (Nagatani, 1997).

### EXAMPLE 11

### Quantification of Hsp101 Levels in Kanamycin-resistant Plants

Transformed kanamycin-resistant plants (T1-generation) were grown on GM plates containing 50 mg/L kanamycin {germination medium per liter: 1×Murashige and Skoog medium (Sigma), 1.0 ml M&S vitamins (Sigma), 10 mg sucrose, pH 5.7 with KOH, 2 mg phytagel from Sigma} at 22° C. in Percival incubators I-35LVL and E-30B under continuos light (150-300 μmol m⁻² s⁻¹). Fourteen-day-old plants were exposed to 38° C. for 90 min in the light. Prior to heat treatment two plants of each genotype were frozen in liquid nitrogen for analysis of Hsp101 expression at 22° C. After heat treatment two plants of each genotype were taken to assess Hsp101 expression after heat stress. Total proteins were extracted by grinding individual frozen plants in plant lysis buffer (100 mM Tris-HCl, pH 8.0, 25 mM KCl, 4 mM CaCl₂, 0.05 mg/mL BSA and protease inhibitor cocktail Complete™, EDTA-free from Boehringer Mannheim, 1 tablet for each 50 mL buffer). Insoluble debris was removed by centrifugation at 10,000 g for 5 to 10 min. Protein concentrations were estimated using the Bio-Rad protein-assay (Bio-Rad Laboratories, Hercules, Calif.). Twelve μg of protein from each sample were suspended in 6×sample buffer {300M Tris-HCl, ph 6.8, 12% (w/v) SDS, 60% (v/v) glycerol, 6% (v/v) 2-mercaptoethanol, 0.12% (w/v) bromophenol blue}, electrophoretically separated proteins (10% SDS-PAGE were transferred to Immobilon-P membranes (Millipore Corp., Bedford, Mass.) for immunological analysis. Equal loading was confirmed by Coomassie Blue staining of the membrane. Membranes were reacted with polyclonal antibodies against Hsp101 (generated against an N-terminal fragment of Hsp101 ) and a monoclonal antibody that recognizes constitutive and heat-inducible species of Hsp70 (7.10, Velazquez and Lindquist, 1983). Immunocomplexes were visualized and quantified with I¹²⁵-Protein A, using a Phosphorlmager and ImageQuant® software (Molecular Dynamics, Sunnyvale, Calif.).

Vector controls, plants with reduced levels of Hsp101 (No-AS1-5, Col-SUP1-5) and plants with constitutive expression of Hsp101 at 22° C. (No-C1, Col-C1, Col-C2) were propagated to homozygosity and grown on GM media without kanamycin. Hsp101 levels in these plants (T2 and T3 generation) were quantified as described herein. Transgenic plants generated by tissue culture transformation were backcrossed twice to wild-type No-0 plants prior to analyis.

### EXAMPLE 12

### Methods for Phenotypic Analysis

To observe general plant growth phenotypes, transgenic plants were grown on soil or PNS medium (2.5 mM potassium phosphate at pH 5.5, 5 mM KNO₃, 2 mM MgSO₄, 2 mM Ca(NO₃)₂, 49 μM C₁₀H₁₂N₂NaFeO₈, micronutrients, 5 g /L sucrose) on a 16 hr/8 hr, 24° C./18° C., day/night cycle in a growth chamber with ˜250 μE m⁻² sec⁻¹. Plants were photographed after fourteen days (PNS), three weeks (soil) or five weeks (soil). Similar results were obtained when plants were grown under continuous light at 24° C. To assess growth under stressful conditions antisense and vector control plants were also grown on a 16 hr/8 hr, 30° C./24° C. day/night cycle with ˜250 μE m⁻² sec⁻¹. Germination rates and frequencies for each genotype were monitored by plating ˜150 vector control seeds and ˜50 seeds of each antisense line (No-AS1-5), co-suppression line (Col-SUP1-5) and constitutive line (NO-C1, Col-C1, Col-C2) together on GM plates. Plates were incubated at 22° C. and under continuous light. Germination was scored daily for three days. Similar results were obtained when plates were incubated for three days at 4° C. after plating prior to incubation at normal growth conditions at 22° C. and in continuous light (150-300 μmol m⁻² s⁻¹) and when sterilized seeds were kept at 4° C. for three days prior to plating.

### EXAMPLE 13

### Methods of Induced Thermotolerance Assays with Fourteen-Day-Old Plants

Homozygous vector controls (No-V1 and V2, Col-V1 and V2) and homozygous plants with altered expression of Hsp101 were plated together on GM plates (without kanamycin, 25 mL medium per plate) and grown as described above for fourteen days. Plates were exposed to one of the following heat treatments:1) 38° C. for 90 min (pretreatment); 2) 38° C. for 90 min, followed 45° C. for 2 hr (conditioned), or 3) 45° C. for 15 min, 30 min, 45 min, 60 min, or 2 hr (unconditioned). After heat treatments plates were returned to 22° C. and viability was assessed daily for up to ten days. Results were documented photographically five or six days after heat stress. Hsp101 protein levels were monitored in the same experiment immediately before and after pretreatment as described above.

### EXAMPLE 14

### Methods for Basal Thermotolerance Germination Assay

Vector control seeds and seeds of antisense lines No-AS1-5 were plated together in rows on GM plates and exposed to 47° C. for 2 hr immediately after sterilization and plating (30 min), or 30 hr, 36 hr, 48 hr, or 72 hr after sterilization and plating. Plates were then returned to normal growth conditions (22° C. with continuous light 150-300 μmol m⁻² s⁻¹). Seed development was scored two, five, and ten days after heat treatment and documented photographically after ten days. Similar results were obtained with sterilized seeds cold treated (4° C. for three days) prior to plating.

For analysis of Hsp101 levels in seeds, 10 mg of seeds for each genotype were ground in 200 μl sample buffer (60 mM Tris-HCl, pH 8.0, 60 mM DTT, 2% (w/v) SDS, 15% (w/v) sucrose, 5 mM ε-amino-N-caproic acid and 1 mM benzamidine). Protein concentration was estimated with a Coomassie Blue binding assay. Proteins (0.5 or 2.5 μg) were separated by 10% SDS-PAGE. For analysis of small HSPs, Hsp21 (Osteryoung et al.,1993) and Hsp17.6 (Wehmeyer et al., 1996), the same samples were separated by 15% SDS PAGE. Chemiluminescence (ECL, Amersham) was used for visualization.

### EXAMPLE 15

### Methods for Basal Thermotolerance Assays of Three-Day-Old Seedlings

Vector control seeds and seeds of constitutive expression lines No-C1, Col-C1, and Col-C2 were plated on GM plates and grown for 72 hr as described above. Plates were then directly exposed to 47° C. for 30 min, 45 min or 2 hr before being returned to normal growth conditions (22° C. in continuous light, 150-300 μmol m⁻² s⁻¹). Viability was assessed daily for up to ten days after heat treatment and results were documented photographically after two days (magnification Olympus DF plan 1×) and after ten days (¼ of plate is shown). Similar results were obtained when seeds were cold treated (4° C. for three days) prior to plating.

### EXAMPLE 16

### Nucleic Acid Hybridization to Detect the Sequences Capable of Coding for the Stress Response Proteins or their Biologically Functional Equivalents

The nucleic acid sequence Hsp100 family information available to a skilled artisan through standard sequence searching methods utilizing databases such as GenBank, or having the following nucleic acid sequences: SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, and/or SEQ ID NO:49. Alternatively a skilled artisan may utilize the following amino acid sequences: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and/or SEQ ID NO:29. These sequences and other related sequences allow for the preparation of relatively short DNA (or RNA) sequences having the ability to specifically hybridize to gene sequences capable of coding for at least the protective domain of the stress proteins. In these aspects, nucleic acid probes of an appropriate length are prepared based on a consideration of the Hsp100-related sequences. The ability of such nucleic acid probes to specifically hybridize to the heat shock proteins lend them particular utility in a variety of embodiments. Most importantly, the probes can be used in a variety of assays for detecting the presence of complementary sequences in a given sample. Other uses are envisioned, including the use of the sequence information for the preparation of mutant species primers, or primers for use in preparing other genetic constructions.

To provide certain of the advantages in accordance with the invention, the preferred nucleic acid sequence employed for hybridization studies or assays includes sequences that are complementary to at least a 14 base nucleotide stretches of a Hsp100 sequence. A size of at least 14 nucleotides in length helps to ensure that the fragment will be of sufficient length to form a duplex molecule that is both stable and selective. Such fragments may be readily prepared by, for example, directly synthesizing the fragment by chemical means, by application of nucleic acid reproduction technology, such as the PCR technology of U.S. Pat. No. 4,603,102, or by introducing selected sequences into recombinant vectors for recombinant production. Larger segments are also within the scope of this invention.

Accordingly, the nucleotide sequences of the invention are important for their ability to selectively form duplex molecules with complementary stretches of the gene. Depending on the application envisioned, varying conditions of hybridization may be employed to achieve varying degree of selectivity of the probe toward the target sequence. For applications requiring a high degree of selectivity, relatively stringent conditions may be employed to form the hybrids, for example, selecting relatively low salt and/or high temperature conditions, such as provided by 0.02M-0.15M NaCl at temperatures of 50° C. to 70° C. These conditions are particularly selective, and tolerate little, if any, mismatch between the probe and the template or target strand.

Alternatively, for some applications, for example, preparation of mutants employing a mutant primer strand hybridized to an underlying template, or to isolate stress protein sequences from related species, functional equivalents, or the like, require less stringent hybridization conditions to allow formation of the heteroduplex. In these circumstances, conditions employed would be, e.g., 0.1 5M-0.9M salt, at temperatures ranging from 20° C. to 55° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, one skilled in the art is aware that hybridization conditions can be readily manipulated, and thus will generally be a method of choice depending on the desired results.

In certain embodiments, it will be advantageous to employ nucleic acid sequences of the present invention in combination with an appropriate means, such as a label, for determining hybridization. A wide variety of appropriate indicator means are known in the art, including radioactive, enzymatic or other ligands, such as avidin/biotin, which are capable of giving a detectable signal. In preferred diagnostic embodiments, an enzyme tag such as urease, alkaline phosphatase or peroxidase, may be employed instead of radioactive or other environmental undesirable reagents. In the case of enzyme tags, calorimetric indicator substrates are known which can be employed to provide a means visible to the human eye or spectrophotometrically, to identify specific hybridization with complementary nucleic acid-containing samples.

In general, it is envisioned that the hybridization probes described herein will be useful both as reagents in solution hybridization as well as in embodiments employing a solid phase. In embodiments involving a solid phase, the test DNA (or RNA) is adsorbed or otherwise affixed to a selected matrix or surface. This fixed, single-stranded nucleic acid is then subjected to specific hybridization with selected probes under desired conditions. The selected conditions will depend on the particular circumstances based on the particular criteria required (depending, for example, on the G+C contents, type of target nucleic acid, source of nucleic acid, size of hybridization probe, etc.). Following washing of the hybridized surface so as to remove nonspecifically bound probe molecules, specific hybridization is detected, or even quantified, by means of the label.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

Bechtold, N., Pelletier, G. (1998). In planta Agrobacterium-mediated transformation of adult Arabidopsis thaliana plants by vacuum infiltration. Methods Mol. Biol. 82, 259-266.

Bizily, S. P., Rugh, C. L., Summers, A. O., Meagher, R. B. (1999) Phytoremidation of methylmercury pollution: merB expression in Arabidopsis thaliana confers resistance to organomercurials. Proc Natl Acad Sci USA 96(12), 6808-6813.

Bizily, S. P., Rugh, C. L., Meagher, R. B. (2000) Phytodetoxification of hazardous organomercurials by genetically engineered plants. Nat Biotechnol. 18(2), 213-217.

Bohnert, H. J., Nelson, D. E., Jensen, R. G. (1995). Adaptations to environmental stresses. Plant Cell 7, 1099-1111.

Boston, R. S., Viitanen, P. V., Vierling, E. (1996). Molecular chaperones and protein folding in plants. Plant Mol. Biol. 32, 191-222.

Eleutherio, E. C., Araujo, P. S., Panek, A. D. (1993). Protective role of trehalose during heat stress in Saccharomyces cerevisiae. Cryobiology 30, 591-596.

Eriksson, M. J., Clarke, A. K. (1996). The heat shock protein ClpB mediates the development of thermotolerance in the cyanobacterium Synechococcus sp. strain PCC 7942. J. Bacteriol. 178, 4839-4846.

Frova, C. (1997). Genetic dissection of thermotolerance in maize. In Physical Stress in Plants, Grillo, S., A. Leone, eds. (Springer-Verlag, N.Y.) pp. 31-38.

Glover, J. R., Lindquist, S. (1998). Hsp104, Hsp70, and Hsp40: a novel chaperone system that rescues previously aggregated proteins. Cell 94, 73-82.

Holmstrom, K.-O., Welin, E. M. B., Palva, E. M. B. W., Tunnela, O. E., Londesborough, J. (1996). Drought tolerance in tobacco. Nature 379, 683-684.

Howarth, C. J., Skot, K. P. (1994). Detailed characterization of heat shock protein synthesis and induced therrnotolerance in seedlings of Sorghum bicolor L. J. Exp. Bot. 45, 1353-1363.

Kasuga, M., Liu, Q., Miura, S., Yamaguchi-Shinozaki, K., Shinozaki, K. (1999). Improving plant drought, salt, and freezing tolerance by gene transfer of a single stress-inducible transcription factor. Nat. Biotechnol. 17, 287-291.

Koncz, C., Schell, J., Redei, G. P. (1992). T-DNA transformation and insertion mutagenesis. In Methods in Arabidopsis Research, Koncz, C., N.-H. Chua, J. Schell, eds. (World Scientific Publishing Co. Pte.Ltd, Singapore) pp. 224-273.

Lee, J. H., Hubel, A., Schöffl, F. (1995). Derepression of the activity of genetically engineered heat shock factor causes constitutive synthesis of heat shock proteins and increased thennotolerance in transgenic Arabidopsis. Plant J. 8, 603-612.

Lee, J. H., Schöffl, F. (1996). An Hsp70 antisense gene affects the expression of HSP70/HSC70, the regulation of HSF, and the acquisition of thermotolerance in transgenic Arabidopsis thaliana. Mol. Gen. Genet. 252, 11-19.

Lee, Y. R., Nagao, R. T., Key, J. L. (1994). A soybean 101-kD heat shock protein complements a yeast HSP104 deletion mutant in acquiring thermotolerance. Plant Cell 6, 1889-1897.

Levitt, J.(1980) Responses of plants to environmental stresses. In Physiological Ecology, Kozlowski, T. T., ed. (Academic Press, New York) pp. 347-448.

Lindquist, S., Kim, G. (1996). Heat-shock protein 104 expression is sufficient for thermotolerance in yeast. Proc. Natl. Acad. Sci. USA 93, 5301-5306.

Matzke, M. A., Matzke, A. J. M. (1995). How and why do plants inactivate homologous (trans)genes? Plant Physiol. 107, 679-685

Mogk, A., Tomoyasu, T., Goloubinoff, P., Rüdiger, S., Röder, D., Langen, H., Bukau, B. (1999). Identification of thermolabile E. coli proteins: prevention and reversion of aggregation by DnaK and ClpB. EMBO J. in press

Motohashi, K., Watanabe, Y., Yohda, M., Yoshida, M. (1999). Heat-inactivated proteins are rescued by the DnaK/J-GrpE set and ClpB chaperones. Proc. Natl. Acad. Sci. USA 96, 7184-7189.

Nakashima, K., Kiyosue, T., Yamaguchi-Shinozaki, K., Shinozaki, K. (1997). A nuclear gene, erdl, encoding a chloroplast-targeted Clp protease regulatory subunit homolog is not only induced by water stress but also developmentally up-regulated during senescence in Arabidopsis thaliana. Plant J. 12, 851-861.

Neuwald, A. F., Aravind, L., Spouge, J. L., Koonin, E. V. (1999). AAA+: A class of chaperone-like ATPases associated with the assembly, operation, and disassembly of protein complexes. Genome Res. 9, 27-43.

Nielsen, E., Akita, M., Davila-Aponte, J., Keegstra, K. (1997). Stable association of chloroplastic precursors with protein translocation complexes that contain proteins from both envelope membranes and a stromal Hsp100 molecular chaperone. EMBO J. 16, 935-946.

Nover, L. (1990). Molecular cell biology of the heat stress response. Part I Naturwissenschaften 77, 310-316.

Osteryoung, K. W., Sundberg, H., Vierling, E. (1993). Poly(A) tail length of a heat shock protein RNA is increased by severe heat stress, but intron splicing is unaffected. Mol. Gen. Genet. 239, 323-333.

Ougham, H. J., Howarth, C. J. (1988). Temperature shock proteins in plants. Symp. Soc. Exp. Biol. 42, 259-280.

Parsell, D. A., Lindquist, S. (1994a). Heat shock proteins and stress tolerance. In The Biology of Heat Shock Proteins and Molecular Chaperones Morimoto, R. I., A. Tissieres, C. Georgopoulos, eds. (Cold Spring Harbor Laboratory Press, New York) pp. 457-494.

Parsell, D. A., Kowal, A. S., Singer, M. A., Lindquist, S. (1994b). Protein disaggregation mediated by heat-shock protein Hsp104. Nature 372, 475-478.

Parsell, D. A., Lindquist, S. (1993). The function of heat-shock proteins in stress tolerance: degradation and reactivation of damaged proteins. Ann. Rev. Genet. 27, 437-496.

Poirier, Y., Nawrath, C., Somerville, C. (1995). Production of polyhydroxyalkanoates, a family of biodegradable plastics and elastomers, in bacteria and plants. Biotechnology (N.Y.) 13(2), 142-150.

Prändl, R., Hinderhofer, K., Eggers-Schumacher, G., Schöffl, F. (1998). HSF3, a new heat shock factor from Arabidopsis thaliana, derepresses the heat shock response and confers thermotolerance when overexpressed in transgenic plants. Mol. Gen. Genet. 258, 269-278.

Ruis, H., Schuller, C. (1995). Stress signaling in yeast. Bioessays 17, 959-965.

Sanchez, Y., Taulien, J., Borkovich, K. A., Lindquist, S. (1992). Hsp104 is required for tolerance to many forms of stress. Embo J. 11, 2357-2364.

Schirmer, E. C., Glover, J. R., Singer, M. A., Lindquist, S. (1996). HSP100/Clp proteins: a common mechanism explains diverse functions. Trends Biochem. Sci. 21, 289-296.

Schirmer, E. C., Lindquist, S., Vierling, E. (1994). An Arabidopsis heat shock protein complements a thermotolerance defect in yeast. Plant Cell 6, 1899-1909.

Shanklin, J., DeWitt, N. D., Flanagan, J. M. (1995). The stroma of higher plant plastids contain ClpP and ClpC, functional homologs of Escherichia coli ClpP and ClpA: an archetypal two-component ATP-dependent protease. Plant Cell 7, 1713-1722.

Singer, M. A., Lindquist, S. (1998). Multiple effects of trehalose on protein folding in vitro and in vivo. Mol. Cell 1, 639-648.

Smirnoff, N. (1998). Plant resistance to environmental stress. Curr. Opin. Biotechnol. 9, 214-219.

Smirnoff, N., Bryant, J. A. (1999). DREB takes the stress out of growing up. Nat. Biotechnol. 17, 229-230.

Solomon, J. M., Rossi, J. M., Golic, K., McGarry, T., Lindquist, S. (1991). Changes in hsp70 alter thermotolerance and heat-shock regulation in Drosophila. New Biologist 3, 1106-1120.

Strittmatter, G., Chua, N. H. (1987) Artificial combination of two cis-regulatory elements generates a unique pattern of expression in transgenic plants. Proc Natl Acad Sci USA 84(24), 8986-8990.

Velazquez, J. M., Sonoda, S., Bugaisky, G., Lindquist, S. (1983). Is the major Drosophila heat shock protein present in cells that have not been heat shocked? J. Cell Biol. 96, 286-290.

Vierling, E. (1991). The roles of heat shock proteins in plants. Annu. Rev. Plant Physiol. Plant Mol. Biol. 42, 579-620.

Weaver, L. M., Froehlich, J. E., Amasino, R. M. (1999). Chloroplast-targeted ERD1 protein declines but its mRNA increases during senescence in Arabidopsis. Plant Physiol. 119, 1209-1216.

Wehmeyer, N., Hernandez, L. D., Finkelstein, R. R., Vierling, E. (1996). Synthesis of small heat-shock proteins is part of the developmental program of late seed maturation. Plant Physiol. 112, 747-757.

Wells, D. R., Tanguay, R. L., Le, H., Gallie, D. R. (1998). HSP101 functions as a specific translational regulatory protein whose activity is regulated by nutrient status. Genes Dev. 12, 3236-3251.

Welte, M. A., Tetrault, J. M., Dellavalle, R. P., Lindquist, S. (1993). A new method for manipulating transgenes: engineering heat tolerance in a complex, multicellular organism. Curr. Biol. 3, 842-853.

Yeh, K. W., Jinn, T. L., Yeh, C. H., Chen, Y. M., Lin, C. Y. (1994). Plant low-molecular-mass heat-shock proteins: their relationship to the acquisition of thermotolerance in plants. Biotechnol. Appl. Biochem. 19, 41-49.

Zahringer, H., Burgert, M., Holzer, H., Nwaka, S. (1997). Neutral trehalase Nth1p of Saccharomyces cerevisiae encoded by the NTH1 gene is a multiple stress responsive protein. FEBS Letters 412, 615-620.

Zolkiewski, M. (1999). ClpB cooperates with DnaK, DnaJ, and GrpE in suppressing protein aggregation. A novel multi-chaperone system from Escherichia coli. J. Biol. Chem. 274, 28083-28086.

One skilled in the art readily appreciates that the present invention is well adapted to carry out the objectives and obtain the ends and advantages mentioned as well as those inherent therein. Plants, seeds, methods, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

We claim:

1. A transgenic plant comprising a genetic construct wherein said construct comprises:
(a) a promoter, wherein said promoter is operatively linked to
(b) a nucleic acid sequence encoding a plant Hsp100 family amino acid sequence.

2. The transgenic plant of claim 1 wherein said plant Hsp100 family amino acid sequence is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29.

3. The transgenic plant of claim 1 wherein said nucleic acid sequence encoding said plant Hsp100 family amino acid sequence is endogenous to said transgenic plant.

4. The transgenic plant of claim 1 wherein said nucleic acid sequence has sequence similarity with a sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, and SEQ ID NO:49.

5. The transgenic plant of claim 1 wherein said transgenic plant is selected from the group consisting of a cereal, a grass, an ornamental plant, a crop plant, a food plant, an oil-producing plant, a synthetic product-producing plant, an environmental waste absorbing plant, an alcohol plant, a medicinal plant, a recreational plant, and an animal feed plant.

6. The transgenic plant of claim 1 wherein said plant is selected from the group consisting of cotton, canola, soybean, corn, wheat, tobacco, sorghum, potato, tomato and Arabidopsis thaliana.

7. The transgenic plant of claim 1 wherein said promoter is selected from the group consisting of a constitutive promoter and an inducible promoter.

8. The constitutive promoter of claim 7 wherein said promoter is selected from the group consisting of a 35S cauliflower mosaic virus promoter, a CaMV-35Somega promoter, an Arabidopsis ubiquitin UBQ1 promoter, and a barley leaf thionin BTH6 promoter.

9. The constitutive promoter of claim 8 wherein said promoter is a 35S cauliflower mosaic virus promoter

10. The inducible promoter of claim 7 wherein said promoter is heat inducible.

11. The heat inducible promoter of claim 10 wherein said promoter is selected from the group consisting of a heat shock protein promoter, a heat shock transcription factor promoter, a chaperonin promoter, an A1494 promoter, a rice genomic metallothionein-like gene (rgMT) promoter a ubiquitin promoter, an FLP promoter, an Oryza sativa metallothionein like gene-2 (OsMT-2) promoter, a Glycine max STI1 (gmsti) promoter, a synthetic heat shock promoter and a TCH gene promoter.

12. A method of increasing stress tolerance of a plant comprising the steps of:
preparing a transgenic plant comprising a genetic construct wherein said construct comprises a promoter, wherein said promoter is operatively linked to a nucleic acid sequence encoding a plant Hsp100 family amino acid sequence; and
exposing said transgenic plant to a heat pretreatment.

13. The method of claim 12, wherein said stress tolerance is thermotolerance.

14. The method of claim 12 wherein said plant is a seedling.

15. A method of producing a crop comprising the steps of:
preparing a transgenic plant in accordance with claim 1, wherein said transgenic plant is a crop plant;
growing said transgenic crop plant in an environment which produces heat stress; and
extracting the crop from said transgenic crop plant.

16. The method of claim 15 wherein said crop plant is selected from the group consisting of cotton, tobacco, corn, sorghum, rice, wheat, peanut, soybean, potato, tomato and canola.

17. A method of producing oil from a plant comprising the steps of:
preparing a transgenic plant in accordance with claim 1, wherein said transgenic plant is an oil-producing plant;
growing said transgenic oil-producing plant in an environment which produces heat stress; and
extracting the oil from said transgenic oil-producing plant.

18. The method according to claim 17 wherein said oil-producing plant is selected from the group consisting of canola, corn, peanut, olive, cotton and soybean.

19. A method of making a synthetic product from a plant comprising the steps of:
preparing a transgenic plant in accordance with claim 1, wherein said transgenic plant is a synthetic product-producing plant;
growing said synthetic product-producing plant in an environment which produces heat stress; and
preparing the synthetic product from said synthetic product-producing plant.

20. A method of making an environmental waste absorbing plant comprising the steps of:
preparing a transgenic plant in accordance with claim 1, wherein said transgenic plant is an environmental waste absorbing plant;
growing said environmental waste absorbing plant in an environment which produces heat stress; and
removing said environmental waste from said environment.

21. A method of making a medicinal plant comprising the steps of:
preparing a transgenic plant in accordance with claim 1, wherein said transgenic plant is a medicinal plant;
growing said medicinal plant in an environment which produces heat stress; and
preparing a medicament from said medicinal plant.

22. A method of making animal feed from a plant comprising the steps of:
preparing a transgenic plant in accordance with claim 1, wherein said transgenic plant is an animal feed-producing plant;
growing said animal feed-producing plant in an environment which produces heat stress; and
preparing the animal feed from said animal feed-producing plant.

23. The method of claim 22 wherein said plant is selected from the group consisting of sorghum, soybean, wheat and corn.

24. A method of making alcohol from a plant comprising the steps of
preparing a transgenic plant in accordance with claim 1, wherein said transgenic plant is an alcohol plant;
growing said alcohol plant in an environment which produces heat stress; and
preparing said alcohol from said alcohol plant.

25. A method of utilizing a recreational plant comprising the steps of:
preparing a transgenic plant in accordance with claim 1, wherein said transgenic plant is a recreational plant;
growing said plant in an environm ent which produces heat stress; and
utilizing said plant for recreational purposes.

26. The method of claim 25 wherein said plant is a grass.

27. As a composition of matter, a seed from a transgenic plant of claim 1.

28. As a composition of matter, a seed from a transgenic plant of claim 2.
